(19) United States
(12) Patent Application Publication
(10) Pub. No.: US 2025/0281100 A1
(43) Pub. Date: Sep. 11, 2025

(54) SYSTEMS AND METHODS FOR FEATURE STATE CHANGE DETECTION AND USES THEREOF

(71) Applicant: PRIMA MEDICAL, INC., Boise, ID (US)

(72) Inventor: Ryan BOKAN, Boise, ID (US)

(21) Appl. No.: 18/858,816

(22) PCT Filed: Apr. 27, 2023
(86) PCT No.: PCT/US2023/020217

§ 371 Date: Oct. 22, 2024

Related U.S. Application Data
(60) Provisional application No. 63/335,700, filed on Apr. 27, 2022.

(51) Int. Cl. A61B 5/367; A61B 5/00; A61B 5/346; G06F 18/22; G16H 20/40

(52) U.S. Cl. CPC A61B 5/367; A61B 5/346; A61B 5/4848; A61B 5/7264; A61B 5/742; G06F 18/22; G16H 20/40; G06F 2218/08

(57) ABSTRACT

Examples are described herein for feature state change detection and used thereof. In some examples, feature signals can be generated based on electrophysiological data captured from a patient. The feature signals can be evaluated to compute feature states. A feature state change can be detected indicative of a change in electrical activity caused at a location on a surface of interest within a patient's body. In some examples, the feature state change is used to identify a potential target site for a therapy. In other examples, the feature state change can be used for treatment suggestion and success recommendations and thus driving a treatment being applied to the patient. Other examples and uses of feature states and/or detected feature state changes are disclosed herein.

### RELATED APPLICATION

This application claims the benefit of U.S. Provisional Patent Application No. 63/335,700, filed Apr. 27, 2022, and entitled “Arrhythmia classification, disease scoring, rhythm state mapping and change detection, unipolar localization, meta procedure endpoints predicting success likelihood and novel software platform for hosting outside devices and software,” which is incorporated herein by reference in its entirety.

### FIELD

This disclosure relates to detecting electrical activity on a surface of interest.

### BACKGROUND

Electrophysiological signals are sensed in a variety of applications, including electroencephalography, electrocardiogramyography, electrooculography, and the like. Electrocardiography measures electrical cardiac information from sensed electrical signals, and can be used to make diagnoses, care decisions, ablation procedure planning, post procedure follow up, etc. Electrocardiographic mapping (ECM) is a technology that is used to determine and display heart electrical information from sensed electrical signals. Cardiac ablation is a procedure used to eliminate or terminate a faulty electrical pathway(s) in a heart which are either prone to developing cardiac arrhythmias or are complicit in (currently contributing to) an existing cardiac arrhythmia. The ablation procedure can be classified by energy source, such as including radiofrequency ablation, radiation ablation, and cryoablation to name a few.

### SUMMARY

### State Change Detector

In an embodiment, one or more non-transitory computer-readable media that include data and machine readable instructions executable by a processor. The data can include electrophysiological data captured from a patient during a treatment. The machine readable instructions can include a feature state quantifier to compute feature states based on feature signals. The feature signals can be generated based on the electrophysiological data. The machine readable instructions can include a state change detector to detect a feature state change indicative of a change in electrical activity on a surface of interest within a patient's body.

### State Change Detection For Map Updating (or Generation)

In another embodiment, a system can include memory configured to store machine readable instructions and data comprising electrophysiological data representing electrophysiological signals captured from a patient during a treatment, and at least one processor configured to access the memory and configured to execute the machine readable instructions. The machine readable instructions can include a feature state quantifier that can include a feature signal generator to compute a number of feature values for features based on respective electrophysiological signals, and combine the feature values for each feature to generate feature signals. The feature state quantifier can include a feature state calculator to compute feature states based on a feature signal segment of a respective feature signal. The machine readable instructions can include a state change detector to detect a feature state change indicative of a change in electrical activity on a surface of interest within a patient's body, and a target generator to output target map data identifying a location(s) of interest on the surface of interest based on the detected feature state change, the underlying disease features at the location where treatment elicited the state change, and/or other locations exhibiting these similar disease features.

In a further embodiment, a computer-implemented method can include receiving, by a processor, electrophysiological data captured from a patient during a treatment of a target site identified prior to a treatment. The target site can correspond to a potential ablation site on a surface of interest within a patient's body. The computer-implemented method can further include generating, by the processor, feature signals based on the electrophysiological data captured from the patient, computing, by the processor, feature states based on the feature signals, detecting, by the processor, a respective feature state change based on an evaluation of the feature states relative to state change detection criteria, and outputting, by the processor, target map data identifying a region(s) of interest on a surface of interest

### Categorize Mapping Points for a Composite Feature State Group

In some examples, feature signals and feature state data can be used for categorizing each mapping point to a composite feature state. For example, a system can determine which composite feature state was present at a time of each map point and the map point can be assigned to a group matched to that composite feature state value. In some instances, a clinician can use this system during catheter mapping to determine when a mapping catheter has recorded a signal long enough for a particular composite feature state group at a given location such that the clinician can move the catheter to another location for mapping. For example, the system can characterize a periodicity of the feature signal by measuring how much time for which the signal feature repeats itself in a given state, and this time value can serve as the minimum time that the clinician should collect mapping data (e.g., signal measurements) at a particular location before moving to the next location. In instance where a mapping system is used to generate (or create) mapping data across a surface of interest (e.g., a heart) for each time instance (e.g., single beat mapping such as ECGI), this can indicate which mapping intervals are used by the system for computing target map data, as disclosed herein. In some examples, during a heart mapping process, the system can indicate on a surface of interest model (e.g., heart surface model) locations that may need further mapping for each composite feature state group detected.

In some examples, the system can generate a target map for each composite feature state group. In this instance, the system can display target maps for a most versus least severe composite feature state groups on a display (e.g., for clinician review). In this example, the system can display target maps for any user chosen or program chosen composite feature state. In other examples, the system can generate an aggregate target map that aggregates data from the target maps across all composite feature state groups. In one example, the system can display target map data whose importance or display is time weighted to how often the composite feature state simultaneous to each mapped point was present. The system can be configurable to display aggregate targets based on one or more of: a time in which that map's composite feature state group is presented over a long time window and severity associated with each presenting composite feature state.

### Localize Rhythms for a Composite Feature State Group

In some examples, a system can use or include a rhythm localizer that can process the feature state data associated with a particular composite feature state group. For example, the rhythm localizer can identify or differentiate (e.g., by highlighting on a model heart surface a region having the highest likelihood to be housing either a focal source or substrate enabling abnormal circuits (e.g., micro or macro reentrant circuits). The rhythm localizer can be applied across all feature states, or can be applied individually to each feature state or feature state group. An overall target map or individual feature state target maps can be generated to include the localization information (e.g., one or more regions and/or X, Y, Z locations having a highest likelihood housing information).

### Reinforcement Learning for Rhythm Localizer for a Given Composite Feature State Group

In some examples, a feature state change detector can be used to auto-label datasets for reinforcement learning of the rhythm localizer. For example, when a treatment point or a group of treatment points are delivered and these treatment points elicit a feature state change, state change data, lesion location information (e.g., X, Y, Z location of a lesion(s)), disease features, and electrophysiological signals (e.g., EP signals) can be stored memory in a database. This database can be used to train a machine learning (ML) model (e.g., which can be implemented as part of feature state calculators that output location information) to predict location information (e.g., the X, Y, Z locations on a surface of interest, such as the heart's surface) that if treated, is most likely to elicit a state change. The ML model can provide this prediction using input data that can include disease features and waveform features computed from or based on mapping data (e.g., whole chamber heart mapping data) or input data that can include feature state data and (e.g. stationary ECG/catheter signal data, respectively) during a feature state at any point during the procedure. The datasets along which the ML model is trained can be trained from all datasets in the database, or alternatively, exclusively based on one or more datasets that match a patient's baseline characteristics (e.g., feature state changes that have been recorded prior).

### Predict Success Likelihood Given Prior Feature State Data and Feature State Change Data that Occurred Prior in the Case

In some examples, at any given time during a treatment (e.g., therapy or procedure), the baseline feature state data can be analyzed in combination with feature state data during treatment, and associated feature state change data, to compute or predict to likelihood that the patient would have long term clinical success if the clinician stopped treating at this moment.

### Compute Feature State Value Goals and the Predicted Success Likelihood Upon Achievement

In some examples, at any given time during the treatment, the baseline feature state data can be analyzed in combination with feature state data during treatment, and the associated feature state change data, to compute or predict feature state value goals and/or feature state change goals whose achievement will correspond with an explicit success likelihood percentage.

Further embodiments, features, and advantages of the invention, as well as the structure and operation of the various embodiments of the invention are described in detail below with reference to accompanying drawings.

### BRIEF DESCRIPTION OF THE DRAWINGS

Embodiments are described with reference to the accompanying drawings. In the drawings, like reference numbers can indicate identical or functionally similar elements. The drawing in which an element first appears is generally indicated by the left-most digit in the corresponding reference number.

FIG. 1 is a block diagram of a feature detector.

FIG. 2 is a block diagram of a target site identification system.

FIG. 3 is a block diagram of an electrocardiographic mapping and target site identification system.

FIG. 4 is a block diagram of a treatment system.

FIG. 5 is an example of a plot of electrophysiological signals.

FIG. 6 is another example of a plot of electrophysiological signals.

FIG. 7A is an example of a baseline feature map.

FIG. 7B is an example of a feature map with one or more detected feature state changes.

FIG. 8 is an example of another disease map.

FIG. 9 is an example of a plot of feature signals.

FIG. 10 illustrates an example of a method for identifying a target site within a body of a patient.

FIG. 11 is an example of a method for predicting treatment success.

FIG. 12 is another example of a method for predicting treatment success.

FIG. 13 illustrates an example of a method for providing a treatment suggestion.

FIG. 14 is an example of a method for updating treatment targets using information from prior state changes.

FIG. 15 is an example of a method for arrhythmia state meta endpoints for treatment success.

FIG. 16 is an example plot of electrophysiological signals that can be processed according to a p-wave localization technique, as disclosed herein.

FIG. 17 depicts an example computing environment that can be used to perform methods according to an aspect of the present disclosure.

FIG. 18 is an example mapping point plot.

FIG. 19 is an example of a map with mapping points.

FIG. 20 is an example of a number of maps with mapping points.

FIG. 21 is an example of a method for predicting procedure success conditions.

FIG. 22 is an example of different types of feature signals that underlie a feature state.

FIG. 23 is an example of different state changes that can be detected or identified by a feature detector.

### DETAILED DESCRIPTION

This disclosure relates to systems and methods for feature state quantification, feature state change detection and uses thereof. ECGs (electrocardiograms) from electrodes (e.g., 12-Lead ECG or catheter electrodes or other sensors in or around the patient measuring electrical heart signals) inside of a heart are used in electrophysiology (EP) labs to diagnose and help plan treatment for arrhythmogenic activity. For example, at an outset of an ablation procedure, an EP study is generally performed to assess a patient's heart functionality as it relates to cardiac electrical activity. Typically, 12-lead ECG pads are placed on the patient's torso, which generate 12-lead ECG signals throughout the ablation procedure. A multi-electrode catheter is typically placed in the Coronary Sinus. Other multi-electrode catheters can be positioned in fixed locations such as, but not limited to, the right atrial free wall. A target volume (or heart volume) is generated by a program (or software) that is logging spatial coordinates (e.g., in three-dimensional (3D) space) of a catheter as a clinician (e.g., a physician, a user, etc.) moves the catheter around the heart (e.g., heart volume). In some existing approaches, the identification of target sites prior to treatment (referred to herein as prior target sites) is a manual process that relies on a clinician's judgment. In other instances, identification of target sites is carried out automatically using software algorithms that analyze the recorded electrophysiological data from the catheter (mapping signals). Some techniques but not all include the use of machine-learning. Thus, the efficacy of treating arrhythmias within the patient is partly dependent on an accuracy at which arrhythmia origin sites are identified.

Once arrhythmia origin sites are identified, the clinician applies a therapy to these sites using invasive or non-invasive ablation techniques. During ablation, such as cardiac ablation, treatment of a target site (e.g., a location) on a patient's heart (e.g., invasively or non-invasively) can at times cause a drastic or subtle change in arrhythmogenic activity on a surface of the patient's heart. The ablation can have a) unmasked new arrhythmia drivers on the patient's heart that had not been previously identified during the treatment site identification stage (e.g., based on baseline mapping information), b) eliminated an arrhythmia driver such that the arrhythmia activation is now different, and/or c) temporarily hindered an arrhythmia driver or ablated near a driving circuit if the arrhythmogenic activity change was transient or subtle in the presentation of the change.

Existing techniques are unable to quantify an arrhythmogenic activity state (e.g, a disorganized arrhythmias such as fibrillation), nor detect changes there to. Therefore, existing techniques are unable to quantitatively provide clinician feedback so that the clinician can gain insight into phenomenon a-c above, and act in response to these insights. Furthermore, existing techniques are unable to detect or determine which treated signals elicited a change in arrhythmogenic activity (e.g., culprit signal properties), nor identify other mapped signal locations that exhibited similar signal properties to that of the culprit properties. Additionally, existing techniques are unable to detect in real-time instances when electrical (arrhythmogenic) activity previously undetected is now unmasked so that the clinician can remap or technology can localize the newly presenting arrhythmogenic activity and adjust a therapy to eliminate or contain this new arrhythmogenic activity. Consequently, clinicians cannot rely solely on electrocardiographic baseline maps in their current form as these maps only depict prior target sites (identified at the outset of the treatment), and are not inclusive of underlying targets that become unmasked as the arrhythmia becomes modified. Additionally, clinicians are limited in feedback by their real time visual interpretation of stationary catheter or ECG signals to determine if an ablation caused an arrhythmogenic change, and if treatment should be redirected. Thus, it is visually impractical to rely solely on the visual interpretation of real-time signals to redirect treatment.

Systems and methods are disclosed herein for feature state quantification, feature state change detection and uses thereof. According to the systems and methods disclosed herein, electrical activity on a surface of interest within a patient's body can be detected. In some examples, the system can quantify feature signals and feature states at each time during the procedure. In fibrillatory arrhythmias, such as Atrial Fibrillation, it is challenging for the user or clinician to determine a status of the arrhythmia given its constantly changing beat to beat dynamics. It is also difficult for the physician to determine how long to place a mapping catheter at a fixed location in order to acquire the necessary amount of signal data such that it spans the entire cycle or interval of the chaotic arrhythmia. A system that can quantify feature states throughout a procedure is capable of providing users with guidance such as how long to map at each location, the status of the current arrhythmia with respect to the beginning of the case and with respect to arrhythmia status goals that are associated with a predicted clinical success likelihood. Furthermore, this feature state quantification allows for a system to classify groups of various feature states, and output disease maps or localization information for each of these groups.

In some examples, the systems and methods disclosed herein can detect a change in the electrical activity (or electrical activation) that can be caused by delivery of a treatment (e.g., energy delivery) at a location on the surface of interest. This change in the electrical activity can be detected as a state change according to the examples disclosed herein. The detected state change can be indicative that a clinician is in proximity to, near, adjacent, and/or is touching (e.g., with an energy delivery device) a diseased circuit on the surface of interest. A diseased circuit as used herein can refer to an abnormal (or faulty) electrical pathway. The abnormal electrical pathway in some examples can be complicit or contribute to an arrhythmia. In some examples, the diseased circuit can include an abnormal circuit, a reentry circuit, and/or other types of arrhythmia circuits. In some examples, subtle and major state changes can be detected according to the examples disclosed herein. Based on which state change is detected, according to the examples disclosed herein, can be used to inform the clinician and drive how the treatment (e.g., therapy) is delivered to the surface of interest.

In some examples, new potential target sites can be identified on the surface of interest based on the detected state change. Treatment can be applied to the new potential target sites to change or modify an electrical pathway (e.g, faulty electrical pathway) on the surface of interest and thus treat (e.g., remove or terminate) unwanted electrical activation. According to the examples disclosed herein, arrhythmogenic activity (or faulty electrical activity) and its corresponding changes can be quantified, displayed, and provided to a user (e.g., the clinician). As such, the clinician can continue treatment at the target site eliciting the arrhythmogenic activity change, move to another target location, and/or remap the heart signals. In some examples, the detected feature state change can be used to identify arrhythmogenic activity on the surface of interest that had not been previously detected prior to treatment. The detected feature state change can be used to provide a map identifying the arrhythmogenic activity on the surface of interest as one or more new potential target sites for treatment. Accordingly, new arrhythmia drivers can be unmasked or detected during the treatment and the clinician can be alerted (e.g., with the map) of the new potential target sites according to the examples disclosed herein.

In some examples, the systems and methods disclosed herein can be used to predict a success of a treatment point (location or node on the surface of interest) and provide treatment suggestions. For example, the systems and methods as disclosed herein can predict how successful treating a target site is based on a similarity of disease features for the target site relative to disease features of prior ablated target sites that caused or contributed to an arrhythmogenic change. In some examples, the system and methods as disclosed herein can provide treatment suggestions indicating whether the clinician should continue applying a therapy to the target site (or area) on the surface of interest currently being treated, to other prior identified target sites, or whether the clinician should terminate the treatment earlier. In some examples, the treatment suggestion can be provided to the clinician visually or audibly.

While examples herein are disclosed in the context of cardiac electrical signals, it is to be understood that the approaches disclosed herein are equally applicable to other electrophysiological signals, such as electroencephalography, electromyography, electrooculography, and the like.

### Feature Detector and Use Thereof

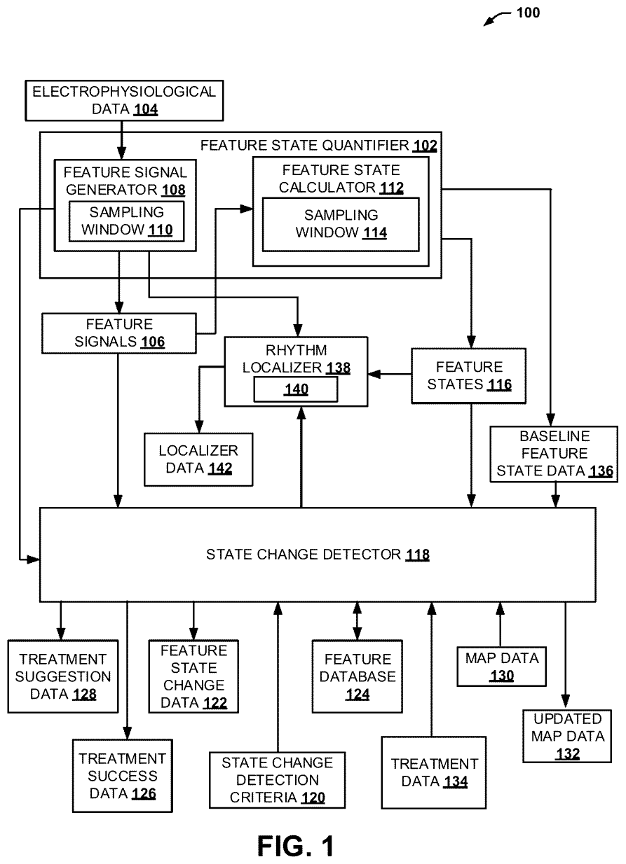

FIG. 1 is an example of a feature detector 100 that can be used to detect feature state changes. In some examples, the detected feature state change can be used for identifying a target site on a surface of interest within a patient's body for treatment, predicting a success of the treatment, and providing treatment suggestions. The feature detector 100 can be implemented as hardware (e.g., circuit and/or devices), software (e.g., a non-transitory medium having machine-readable instructions), or a combination of hardware and software. The feature detector 100 can evaluate electrophysiological signals to identify features of the electrophysiological signals for feature state change detection.

For example, the feature detector 100 can construct or generate a feature signal for each electrophysiological signal. In some examples, the feature detector 100 can provide a feature signal based on a subset of electrophysiological signals. The term “subset” as used herein can refer to a single element, or two more elements. Each feature signal can characterize one or more features that have been identified based on the subset of electrophysiological signals. Thus, in some examples, two or more electrophysiological signals can be processed according to the examples disclosed herein for computing a feature signal. A number of feature signals can be generated for each (or a subset of) electrophysiological signals of the electrophysiological data 104.

Continuing with the example of FIG. 1, the feature detector 100 can include a feature state quantifier 102 that can process electrophysiological data 104 to generate a number of feature signals 106. The electrophysiological data 104 can be stored in a memory (e.g., as disclosed herein) and can be representative of electrophysiological signals obtained by sensors, for example, prior to and/or during the treatment. The sensors can be applied to measure an electrical activity of an anatomical structure of a patient invasively or non-invasively. For example, the sensors may be positioned over a patient's body surface such as a patient's thorax (e.g., for electrocardiography) and/or positioned on a catheter using for capturing or recording the electrical activity from an anatomical structure (e.g., patient's heart) within the patient's body.

Additionally, or alternatively, other sensors (e.g., a twelve lead ECG or the like) can be used to measure the electrophysiological signals from the patient's body. In some examples, the electrophysiological data 104 can include unipolar and/or bipolar electrophysiological signals. Thus, in some instances, a type of electrophysiological signal can depend on a type of sensor being used to measure the electrophysiological signals from the patient's body. In some examples, the electrophysiological signals are acquired in real-time, such as during the treatment. In some examples, the electrophysiological data 104 can correspond to a real time data flow that can be acquired by non-invasive (e.g., body surface) sensors (e.g., over a number of channels) or acquired invasively by one or more sensors on one or more catheters for capturing or recording the electrical activity from the anatomical structure, for example, during a procedure. The procedure can include an electrophysiological study, and/or a treatment procedure that can include cardiac ablation.

In some examples, the electrophysiological data 104 includes electrophysiological measurements acquired over a period of time prior to a procedure (or treatment), such as by a Holter monitoring system, a subcutaneous implant, an implantable device, or the like. In other examples, the electrophysiological data 104 can be acquired invasively, such as by one or more electrodes positioned within the patient's body (e.g., on a lead or a basket catheter during an EP study, during the treatment procedure, or the like). In yet other examples, the electrophysiological data 104 includes both non-invasively acquired and invasively acquired electrical signals. Accordingly, the electrophysiological data 104 can include electrophysiological signals captured before and/or during the treatment.

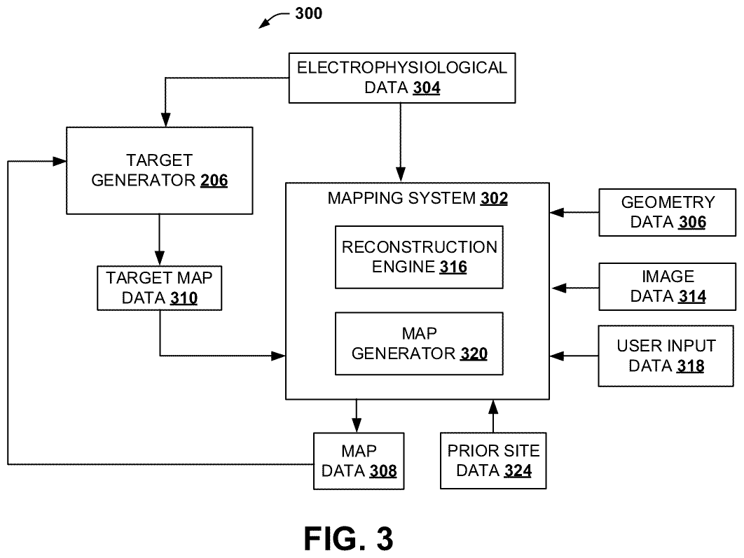

As disclosed herein, the electrophysiological data 104 can be used for reconstruction of the electrical activity across the surface of interest within the patient's body. For example, the electrophysiological data 104 can be provided to a mapping system (e.g., a mapping system 302, as shown in FIG. 3). The mapping system can be configured to reconstruct electrophysiological signals on the surface of interest, in some instances by solving an inverse problem, based on the electrophysiological signals and geometry data for the surface of interest. In some examples, the mapping system generates one or more maps that are displayed to a user (e.g., on a screen or other display device, including stationary and/or portable devices) and at least one of the one or more maps can include target sites identified according to the examples disclosed herein.

The feature state quantifier 102 can include a feature signal generator 108 that can determine a feature based on the electrophysiological data 104. The feature signal generator 108 can sample one or more electrophysiological signals of the electrophysiological data 104 to calculate a respective feature. The feature signal generator 108 can use a sampling window (e.g., of a defined length, for example, four (4) seconds, or a different window length), referred to as an electrophysiological sampling (ES) window 110, to sample a respective electrophysiological signal (e.g., in some instances, acquired over an extended period of time, for example, fifteen (15) minutes). Thus, the respective feature can be computed by the feature signal generator 108 based on a signal segment of the respective electrophysiological signal, which can be referred to as an electrophysiological signal segment. In some examples, the respective feature can be computed based on electrophysiological signal segments from different electrophysiological signals.

In some examples, an electrophysiological signal can include a number of active portions and non active portions. An active portion can correspond to a portion of a signal that has deviated from a signal's baseline (e.g., a signal's baseline noise floor). The signal's baseline of the signal can be the non active portion. The feature signal generator 108 can calculate for each active portion a sub-feature value corresponding to a feature and construct a feature signal, as disclosed herein. In some examples, the feature signal generator 108 can evaluate the electrophysiological signals to identify active and non active portions. The feature signal generator 108 can flag the active portions as electrophysiological signal segments for further processing (e.g., generating of the feature signals 106).

In some examples, the feature signal generator 108 can identify or detect a prominent zone based on the electrophysiological data 104. A prominent zone refers to a specific area or segment of an electrical signal, such as an electrocardiogram (ECG), that has one or more deflections or waves (or a group) with similar characteristics, such as a height, a width, and/or proximity to neighboring waves. To identify the prominent zone, the feature signal generator 108 can group the deflections together based on these characteristics. Then, a candidate wavelet that stands out the most from its preceding and succeeding zones can be confirmed as the prominent zone. A wavelet refers to a single deflection or wave within an electrical signal. For example, the electrical activity on the surface of interest of the patient's heart can be captured and plotted to produce a graph of the electrical activity on the surface of interest. The graph is made up of several waves that can represent different phases of a heart's electrical cycle.

Each wave can be typically made up of several deflections, such as a positive peak (called a “P wave” in some instances) and a negative peak (called a “Q wave” or “S wave” in some instances). In the context of identifying prominent zones in an ECG, a wavelet refers to one of these individual deflections, or peaks, within a wave. By grouping together wavelets that have similar characteristics, such as amplitude, slope, and/or proximity to neighboring wavelets, a prominent zone can be identified within an ECG signal by the feature signal generator 108. As such, in some instances, the feature signal generator 108 can identify a prominent zone by identifying a group of waves with larger amplitudes, steeper slopes, and/or closer proximity to each other than neighboring waves. The feature signal generator 108 can flag each prominent zone as an electrophysiological signal segment of the ECG signal for further processing (e.g., the generating of feature signals 106).

In some examples, the feature signal generator 108 can apply or use a repeatability threshold. A repeatability threshold refers to a statistical measure that can be used to determine a consistency of a candidate wavelet, in relation to its preceding and following zones, across a defined sampling window. The feature signal generator 108 can use the repeatability threshold to evaluate or determine how consistent the candidate prominent wavelet is across a certain period of time. By using the repeatability threshold, the feature signal generator 108 can confirm that the candidate wavelet is not just a one-time occurrence or a random fluctuation of electrophysiological signal, but rather a consistent and meaningful feature. As such, once a candidate wavelet has been identified as a potentially prominent zone based on the amplitude, the slope, and/or the proximity to neighboring waves, the repeatability threshold can be used by the feature signals generator 108 to confirm that the candidate wavelet is prominent. The term “prominent” as used herein in relation to a candidate wavelet can refer to waves or deflections that stand out from a surrounding signal and can be indicative of meaningful features or events in a heart's electrical cycle.

The feature signal generator 108 can identify candidate wavelets by applying various criteria, such as amplitude, slope, and/or proximity to neighboring waves, to determine whether a particular wave or deflection meets the definition of “prominent” based on the repeatability threshold. Accordingly, if a candidate wavelet meets the criteria and is prominent (e.g., consistent) over a defined sampling window (e.g., the sampling window 110, as an example), it may be considered a true prominent wavelet. The feature signal generator 108 can flag each true prominent wavelet as an electrophysiological signal segment for further processing (e.g., generating of the feature signals 106).

The feature signal generator 108 can compute the respective feature based on an electrophysiological signal segment from one or more electrophysiological signals of the electrophysiological signals. Each feature can be used by the feature signal generator 108 to construct a respective feature signal of the feature signals 106. For example, the feature signal generator 108 can plot each feature value for the respective feature computed to provide the respective feature signal. Thus, each feature (value) can be calculated or computed based on one or more characteristics of one or more sampled electrophysiological signal segments. Example features that can be calculated or determined by the feature signal generator 108 can include, but not limited to, a cardiac cycle-length (CL), a synchrony, an ECG morphology classification, a signal amplitude, a cardiac rhythm classification, a peak timing value, in particular R-peak timing value, a peak variability, in particular R-peak variability or a quantitative change between beats, in particular consecutive beats, for example, a mean square error value. A synchrony feature can characterize beat to beat, or window to window, consistency in which activations (e.g., active zones) from two sensors some distance apart are separated in time. Thus, in some examples, the synchrony feature can characterize an interrelationship between two or more electrophysiological signals (or channels over which the signals are captured).

Accordingly, the feature signal generator 108 can slide the ES window 110 (or a respective ES window) across the one or more electrophysiological signals. For each electrophysiological signal segment sampled by the ES window 110, the feature signal generator 108 can compute the respective feature (value) for generating a respective one of the feature signals 106. As such, the feature signal generator 108 can combine computed features (values) to provide the feature signals 106. Each feature signal can be stored in memory (e.g., as disclosed herein) and associated with feature identification information identifying the respective feature (e.g., cardiac CL).

In some examples, the feature signal generator 108 can calculate the respective feature based on electrophysiological signal segments from a similar or same electrophysiological signal over a period of time, or from different electrophysiological signals, which can at least partially overlap in time. For example, the feature signal generator 108 can compute a sub-feature value (a sub-feature) for each identified electrophysiological signal segment and combine computed sub-feature values to provide the respective feature. In other examples, the feature signal generator 108 can use statistical techniques to process the computed sub-feature values for computing the respective feature. Thus, the feature signal generator 108 can compute features based on a number of electrophysiological signal segments from the same or different electrophysiological signals to provide the respective feature signal. As such, the feature signals 106 can include one or more feature signals computed based on electrophysiological signal segments of a similar or different electrophysiological signal.

The feature state quantifier 102 can include a feature state calculator 112. The feature state calculator 112 can determine or compute a number of feature states based on the feature signals 106. The feature state calculator 112 can use a feature sampling (FS) window 114 (e.g., of a defined length, for example, one (1) second, or a different defined length) to sample each feature signal (or a subset thereof) of the feature signals 106. A portion of a respective feature signal on which a feature state is computed by the feature state calculator 112 can be referred to herein as a feature signal segment. The term “feature state” as used herein can refer to a signal property computed or derived based on a feature signal segment. As such, a feature state computed according to the examples disclosed herein can characterize the signal property of a portion of a respective feature signal.

As feature states are computed for the respective feature signal (e.g., on different portions of the respective feature signal over time) the signal properties can change over time (e.g., in response to treatment of the surface of interest). In some examples, the signal properties are statistical properties. In some examples, signal properties computed or derived based on the feature signals 106 herein can be referred to as synthetic signal properties to differentiate signal properties (e.g., features) computed based on electrophysiological signals. Therefore, each computed feature state can characterize a synthetic signal property of a sampled feature signal segment. By way of further example, the feature state calculator 112 can compute a number of feature states 116 for the respective feature signal over time. Example feature states can include, but not limited to, a rate of change, a frequency, a mean, a standard deviation, an interquartile range (IQR), and/or median of the respective feature signal during the FS window 114. In some examples, the respective feature state can include an average deviation computed for the respective feature signal during the FS window 114. For example, the feature state calculator 112 can slide the FS window 114 (or a respective FS window) across each feature signal of the feature signals 106 to provide the feature states 116, as shown in FIG. 1, which can be stored in memory (e.g., as disclosed herein). As disclosed herein, the feature states 116 computed for the respective feature signal can be evaluated to detect a feature state change.

The feature detector 100 includes a state change detector 118 for feature state change detection (e.g., detecting a change in synthetic signal properties of the respective feature signal over time). In some examples, the state change detector 118 can detect a feature state change for each feature signal (or a subset thereof) of the feature signals 106 based on the feature states 116 and state change detection criteria 120. The state change detector 118 can provide feature state change data 122 in response to detecting the feature state change. The state change detection criteria 120 can include a state change threshold. In some examples, the state change detection criteria 120 can include a respective state change threshold for each synthetic signal property that can be computed based on the feature signals 106. For example, the state change detector 118 can evaluate each feature state of the feature states 116 for each feature signal relative to the state change threshold to determine whether a feature state change has occurred. If a feature state is greater than or equal to the state change threshold this can be indicative of a feature state change. One or more thresholds of the state change detection criteria 120 can be derived based on a behavior of feature states recorded at baseline (e.g., prior to treatment). The one or more thresholds can be derived or computed as a function of these behaviors (e.g., a change 10% above a max observed at baseline).

The detected state change by the state change detector 118 can be indicative that a clinician is in proximity to, near, adjacent, and/or is touching (e.g., with a treatment device) a diseased circuit (or area) on the surface of interest. In some examples, the detected state change is a subtle state change or a major state change. A subtle state change can suggest the treatment effected a subset of the arrhythmogenic substrate sustaining a circuit, but since it is not a major (e.g., a rhythm localizer 138 still shows the same region despite this subtle state change based on the localizer data 142 once mapped onto a model of the surface of interest), the clinician can continue searching and treating in the immediate area to completely eliminate arrhythmogenic activity in that location. In some examples, when a major state change occurred (e.g., the rhythm localizer 138 shows different information, that is, the localizer data 142 once mapped onto the surface of interest model identifies new regions or locations on the surface of interest), the clinician can move to another target (or treatment) site. In some examples, the detected state change (e.g., over a period of time) is a transient state change (e.g., returns back to previous state some short time later) can suggest to continue treating diseased signals in an area or zone until permanent state changes occur (e.g., over a period of time), which can include subtle or major state changes. The subtle state change can provide an indication or suggestion to the clinician to keep treating the region or area until major occurs. The major state change can provide an indication or suggestion to the clinician to move to another region or area on the surface of interest.

In some examples, to differentiate whether the state change is a subtle or major state change, the state change detector 118 can evaluate the detected state change relative to a change type threshold. Based on a distance from the change type threshold this can be used to indicate whether the state change is a subtle state change or major state change. For example, if the detected state change is within a first percentage or value from the change type threshold this can be indicative that the detected state change is a major state change, and if the detected state change is within a second percentage or value from the change type threshold this can be indicative that the detected state change is a subtle state change. Accordingly, based on which state change is detected can be used to inform the clinician and drive how the treatment is delivered to the surface of interest.

In some examples, new potential target sites can be identified on the surface of interest based on the detected state change. Treatment can be applied to the new potential target sites to change or modify an electrical pathway or circuitry of electrical activity on the surface of interest and thereby disrupt prior electrical activation. According to the examples herein, arrhythmogenic activity (or faulty electrical activity) and its corresponding changes can be quantified, displayed, and provided to the user (e.g., a clinician). As such, the user can continue treatment at the location eliciting the arrhythmogenic activity change, move to another target location, remap the heart signals, etc. In some examples, the detected feature state change can be used to identify arrhythmogenic activity on the surface of interest that had not been previously detected (e.g., before treatment). The detected feature state change can be used to provide a map identifying the arrhythmogenic activity on the surface of interest as one or more potential target sites for treatment. Accordingly, new arrhythmia drivers can be unmasked or detected during the treatment and the clinician can be alerted (e.g., with the map) of the new potential target sites.

For example, during the treatment, a therapy (e.g., an ablation) can be delivered to a target site identified prior to treatment, that is, a prior target site. Electrophysiological signals at about a time or at a time after delivery of the therapy can be captured and processed by the feature signal generator 108 to provide the feature signals 106. The feature signals 106 can be processed by the feature state calculator 112 to compute feature states 116. The feature states 116 can be processed by the state change detector 118 to detect one or more feature state changes therein (if such changes exist). The delivering of the therapy to the prior target site can cause changes in feature signal segments, which can be reflected by the feature states 116 for these feature signal segments. Thus, for example, if a given feature state is equal to or greater than the state change threshold this can be indicative of a feature state change. Thus, as a prior computed feature state for a feature signal segment changes to a new value for a subsequent (or downstream computed) feature signal segment and that new value exceeds the state change threshold this can be indicative of the feature state change.

In some examples, the state change detector 118 can evaluate computed feature states for feature signal segments to detect the feature state change. For example, the state change detector 118 can compute a difference between subsequent computed feature states and if the difference exceeds the state change threshold this can be indicative of the feature state change. In some examples, the state change detector 118 can compute the difference between feature states that are not neighboring. Thus, the state change detector 118, in some instances, can detect the feature state change when a feature state change is sufficiently large over a period of time so that a feature state change difference exceeds the state change threshold.

In some examples, baseline feature state data 136 can be used by the state change detector 118 to detect the feature state change. In some examples, the feature detector 100 can receive electrophysiological data characterizing electrical activity on the surface of interest captured before treatment, which can be processed according to the examples disclosed herein to provide the baseline feature state data 136 by the feature state calculator 112. Thus, in some examples, the feature signal generator 108 can process the electrophysiological data 104 characterizing electrical activity on the surface of interest captured before treatment to provide baseline feature signals, which the feature state calculator 112 can process to provide the baseline feature state data 136. In some examples, the baseline feature state data 136 is used to compute the one or more thresholds of the state change detection criteria 120. Electrophysiological signals captured before treatment can be referred to as baseline electrophysiological signals in the examples disclosed herein.

In some examples, the state change detector 118 can provide the feature state change data 122 that can indicate that a feature state change has occurred for a synthetic signal property of the respective feature signal, identify a feature (e.g., cardiac CL feature) associated with the respective feature signal, and/or a feature state change time stamp (e.g, specifying a time at which the feature state change was detected or that the feature state was equal or greater than the state change threshold). For example, if a given feature state (e.g., a mean) for the feature signal segment changes so as to exceed the state change threshold (e.g., a mean threshold), the feature state change data 122 can indicate that a feature state change has occurred for the given feature state and identify the feature (e.g., the cardiac CL feature) associated with the respective feature signal. Thus, the feature state change data 122 can identify each feature that changed states corresponding to a synthetic signal property change, in some instances, caused by delivery of therapy to a target site.

In some examples, the state change detector 118 can identify a subset of features of features that had a greatest state change and these identified subset of features can be provided as or part of the feature state change data 122. For example, the state change detector 118 can identify features that are associated with synthetic signal properties that had a greatest feature state change. The state change detector 118 can evaluate feature state changes for these features relative to the state change threshold to identify features that exceed by a given value or percentage the state change threshold as having the greatest state change. For example, if the state change detector 118 determines that the synthetic signal property for a feature signal segment associated with a CL feature was identified as having the greatest state change, the state change detector 118 can provide the feature state change data 122 identifying the CL feature.

In some examples, feature states and feature identification data (associated with the feature states) can be stored as part of a feature database 124, for example, for use on other patients, or the same patient, to identify reconstructed electrophysiological signal at locations on the surface of interest, such as for target site treatment. In some instances, the feature states and the feature identification data can be used for biasing the state change detector 118 so that features having the greatest state change associated with a location of the target site are given more weight for a similar location on the surface of interest (e.g., patient's heart surface) of a different patient. As such, in some examples, the state change detector 118 can be biased so that synthetic signal properties associated with features exhibiting the greatest feature state change are given more weight for a similar location on the surface of interest of other patients for which the feature detector 100 is used.

In some examples, the state change detector 118 can identify a number of feature states for synthetic signal properties associated with different features from signals during prior mapping that, when treated, can cause a state change. Furthermore, this information can be used downstream to identify new target sites with mapping signals that best correlate/resemble those features that caused the state change. Each feature state can be weighted and the weighting result can be used for determining a final feature state for features for a given location on the surface of interest. In some examples, the weights can be user defined, or provided as weights from a historical patent database whose state change instances are most correlated to success. In some examples, the historical weights are stored in the feature database 124, as shown in FIG. 1.

In some examples, the state change detector 118 can use the feature database 124 to identify the subset of features having the greatest state change to provide the feature state change data 122. The feature database 124 can identify a number of historical or baseline feature state changes for a number of features based on prior electrical activity on which the features were computed. For example, the feature database 124 can identify different features, a feature state change (e.g., value) for each feature, and in some instances a location of one or more electrophysiological signals on a surface of interest used for computing each feature. For example, the state change detector 118 can compare each feature state change for each feature to the feature database 124 to identify the subset of features of the features that had the greatest state change.

In some examples, the state change detector 118 can evaluate feature state changes for each feature signal to identify features that exhibit a greatest state change. For example, the state change detector 118 can determine a first feature state change for a feature signal (over a first period of time). The state change detector 118 can determine a second feature state change for the feature signal (over a second period of time). The state change detector 118 can compute a difference between the first and second feature state changes. The greater the difference between the first and second feature state changes can correspond to or be representative of an increase in likelihood that the treatment caused the change and can be assumed that the clinician is closer to treatment success (e.g., ablation success, for example, eliminating the faulty electrical pathway). In some examples, a target success threshold can be used for determining treatment success. The target success threshold can be determined based on prior treatments, and in some instances, be stored as part of the feature database 124. The target success threshold can indicate complete success (e.g., in eliminating the arrhythmogenic activity being targeted by the treatment), and a distance from the target success threshold can be quantified by the state change detector 118 to specify how close the clinician is to success. For example, the state change detector 118 can evaluate the difference between the first and second feature state changes to the target success threshold to determine a likelihood of treatment success.

The state change detector 118 can output treatment success data 126 indicating the likelihood of how successful the treatment is in eliminating the arrhythmogenic activity at a target site. The state change detector 118 can quantify (e.g., as a number, percentage, etc.) the likelihood of how successful the treatment is in eliminating the arrhythmogenic activity at the target site to provide the output treatment success data 126. In some examples, the treatment success data 126 can be rendered on a display (e.g., as disclosed herein) and thus, in some examples, can be provided to the clinician in real-time (e.g., during an on-going treatment).

In some examples, the state change detector 118 can determine a period or an amount of time that a feature state maintains a value or deviates (e.g., within a given value range or percentage) from the value, which can be referred to as a feature state occupied time. For example, the state change detector 118 can determine for a feature state of a respective synthetic signal property for a given feature that the feature state has a value of five (5) or within a range or percentage of this value for a given amount of time. The state change detector 118 can compare the feature state occupied time to a feature state time reference value. The state change detector 118 can output the treatment success data 126 based on the comparison of the feature state occupied time relative to a feature state time reference value. As the feature state occupied time exceeds the feature state time reference value from prior this can be indicative of a greater likelihood of a successful therapy.

The state change detector 118 can provide the treatment success data 126 characterizing the likelihood of successful therapy based on the comparison of the feature state occupied time relative to the feature state time reference value (e.g., a distance between the feature state occupied time and the feature state time reference value). In some examples, the feature database 124 (or a different database) can identify different feature state occupied times for different feature states. The state change detector 118 can use the feature state occupied time for the given feature (from the feature database 124) for comparison with the feature state time reference value to provide the treatment success data 126.

In some examples, the state change detector 118 can output treatment suggestion data 128 in response to detecting the feature state change for a respective feature. The respective feature can be identified in some instances based on historical feature data, which can be stored in the feature database 124. For example, a respective feature can be selected that exhibits a greatest or best likelihood of arrhythmogenic activity detection. The state change detector 118 can determine how close the feature state change (e.g., value) is relative to a feature state change value and provide the treatment suggestion data 128 based on the determination. For example, during the treatment, as therapy (e.g., ablation) is being applied to target sites, the state change detector 118 can output the treatment suggestion data 128 that can suggest (e.g., with text, colors, etc.) whether the clinician should continue with applying the therapy.

In some examples, the suggestion can be represented with a graphical element(s) on a display as a set of colors that change as the feature state change exceeds the feature state time reference value (or in some instances, the one or more thresholds of the state change detection criteria 120). Thus, color suggestions (or other types of visual cues) can be used to suggest to the clinician on whether the clinician should continue with the treatment of the arrhythmogenic activity associated with target sites based on a distance between the feature state change and the feature state change reference value. Thus, as the feature state change nears the feature state change reference value, the display can be provided with changing colors that are indicative of the likelihood that the arrhythmogenic activity associated with the target sites may be eliminated (corresponding to a suggestion). In other examples, an audible alert can be used to provide an indication of the likelihood that the arrhythmogenic activity associated with the target sites may be eliminated. For example, colors similar to a traffic light, such as red, yellow, and green can be used to provide the suggestion on whether to continue with the treatment. For example, the red color can indicate that treatment should likely be terminated and thus indicating that there is a high likelihood that the arrhythmogenic activity associated with the target sites has been eliminated.

The treatment suggestion data 128 can be rendered on a display (e.g., as disclosed herein) and thus, in some examples, can be provided to the clinician in real-time (e.g., during an on-going treatment). By providing the treatment suggestion data 128 on the display during the treatment can allow the clinician to terminate the therapy before applying therapy to all identified target sites if the treatment suggestion data 128 suggests that there is a high likelihood that the arrhythmogenic activity associated with the target sites has been eliminated. Thus, the state change detector 118 can provide the treatment suggestion data 128 when there is a high likelihood that an arrhythmia driver has likely been eliminated, reducing a need to ablate other target sites on the surface of interest.

In some examples, the state change detector 118 can receive map data 130. For example, the map data 130 can include a map that can represent reconstructed electrophysiological signals (of the electrophysiological data 204) spatially and temporally on the surface of interest within the patient's body. In some instances, the map data 130 includes the map of the surface of interest without the electrophysiological signals shown therein and other data shown therein, for example, as disclosed herein. In some examples, the state change detector 118 can update the map data 130 to provide updated map data 132 with disease scores for each point (location) (or subset thereof) on the surface of interest that can be representative of a likelihood of diseased tissue at that point on the surface of interest (e.g., a likelihood that treatment at those point(s) (or sites) on the heart surface will elicit a state change). The surface of interest can be of an anatomical structure within a patient's body. Thus, in some examples, the map data 130 can include the anatomical structure.

In some examples, each location on the surface of interest can have a disease score. The disease score can be a composite score of all features for that location computed according to a disease score equation (or function). A table can be associated with each point (e.g., location) on the surface of interest. Thus, in some examples, each X, Y, Z location on the surface of interest can have an associated table, which can be stored in memory. While examples are disclosed herein in which a table is used, in other examples, a matrix can be constructed, or a different data organizational scheme can be used for tracking data/values, as disclosed herein. Each row of the table can identify a respective feature (e.g., CL, low voltage, active zone, etc.) where a column of the table can identify a feature value for that feature. For example, if 300 features are used, the table for a given point on the surface of interest can have a table with 300 features and 300 corresponding feature values.

Each feature of each row can be associated with a weight value, which can be adjusted to control an influence or contribution of that feature at a given location on the surface of interest. For example, after treatment of the given location on the surface of interest, the feature signal generator 108 can identify an electrophysiological signal segment of a respective electrophysiological signal at a location on the surface of interest and compute a feature value and thus a feature. The state change detector 118 can receive the feature value for each feature in the table from the feature signal generator 108 for a corresponding electrophysiological signal segment at a respective point on the surface of interest. The feature signal generator 108 can quantify each feature for all points on the surface of interest and thus provide corresponding feature value data to the state change detector 118 for updating a respective table for each point on the surface of interest.

The state change detector 118 can evaluate each table to identify features for each location on the surface of interest with feature values having a greatest feature value change. If two or more different features are identified, a select feature can be selected with a greatest feature value change. A weight for the selected feature can be adjusted so the selected feature is given more weight. A new disease score can be computed by the state change detector 118 for each point using the adjusted weight for the selected feature for each point on the surface of interest. For example, for each point, the state change detector 118 can employ the disease scoring equation to compute a composite score, which can correspond to the disease score. In some examples, the disease scoring equation adds up each feature value for a respective feature (multiplied by a corresponding weight) using the table to compute the composite score. In some examples, at an outset of or before the treatment, the weights for each feature in the table for each point on the surface of interest can be selected or set based on the feature database 124. The feature database 124 can specify a number of baseline (or initial) weights for features for points on the surface of interest that have been determined based on prior patients.

After treatment of a given location, this can cause a change in feature states of one or more features at one or more locations on the surface of interest. For example, if we have two state change values that occurred for two different features in the table of each location of the one or more locations on the surface of interest this can be indicative that one of these two features is likely more predictive of a state change in the patient. The feature of the two features having the greatest feature state change can be identified in the table by the state change detector 118 and a weight for this identified feature can be adjusted so treatment of the given location or a different location on the surface interest at a different time gives more weight to the identified feature. The state change detector 118 can for each of the one or more locations on the surface of interest recalculate a new disease score with the identified feature being given more weight, which can be used to provide the updated map data 132, as shown in FIG. 1.

By way of further example, if the CL feature prior to treatment of the given location is the identified feature, the weight for the CL feature can be adjusted in each table for each of the one or more locations so that the new disease score is computed based on a higher weighted CL feature. The state change detector 118 can provide the updated map data 132 with disease scores at the one or more locations with having the CL feature weighted greater. Accordingly, the updated map data 132 can highlight areas of the surface of interest that have a higher weighted CL feature because the disease score has been computed with a greater weight than before to provide the updated map data 132. In some examples, the state change detector 118 represents the disease scores with a given color from a color wheel to differentiate different diseased locations (or areas) on the surface of interest.

In some examples, the state change detector 118 can receive treatment data 134 that can be indicative of a time at which a treatment (e.g., a therapy, such as ablation) was applied to the surface of interest, for example, at a given location. The state change detector 118 (or the feature state calculator 112) can determine a time at which the feature state change was detected. The state change detector 118 can determine a time difference based on the time at which the treatment was applied, and the time at which the feature state change was detected. The state change detector 118 can bias the identified feature based on the time difference. For example, different timing difference weights can be adjusted for different time differences, and a given timing difference weight of the different timing difference weights can be used for adjusting the identified feature (value).

By biasing the identified feature using the time difference can reduce an impact of false positive detected feature state changes being used. For example, if a feature state change is detected by the state change detector 118 after a given amount of time (e.g., after about thirty (30) second) this can be a natural state change, that is, a non-therapy induced state change. By using the time difference and identifying a time difference weight of the different time difference weights for biasing the disease score can reduce or mitigate the impact of false positive feature state changes on the map. This is because the weighting of the identified feature is a function of a time delay between treatment and observed effect and the time delay can be factored into generating the updated map data 132. The map of the updated map data 132 can be updated periodically, selectively (e.g., in response to user input), or in response to detected treatment of a location on the surface of interest. In some examples, the time difference weight identified (its value) can be provided to the disease scoring equation, which can use this value for generating the disease score.

In some examples, the state change detector 118 can provide the updated map data 132 based on the feature state change data 122. For example, the updated map data 132 can include the map with locations identified with a graphical element (referred to as a state change graphical element) indicating where a state change occurred. In some examples, the graphical elements are bulbs. In some examples, the bulbs are associated with locations at which treatment (e.g., ablation) has been applied. A color can be associated with each state change graphical element to provide an indication of a severity of the state change at a corresponding location on the surface of interest. For example, a dark maroon color can be used to indicate a high state change at a location on the surface of interest, whereas a pink color can be used to indicate a low state change at another location on the surface of interest. In some examples, each identified location on the surface of interest by a corresponding state change graphical element that is sufficiently severe (e.g., exceeds some color threshold or different threshold) can be used for identifying a new potential target site that had not been identified at an outset of the treatment.

In some examples, the feature detector 100 can provide the updated map data 132 with the map identifying electrophysiological signals at locations on the surface of interest similar to an anchor signal. For example, a given state change can be identified at a given location on the surface of interest based on an electrophysiological signal (e.g., a segment thereof) captured at the given location. That is, the state change detector 118 can use the feature state change data 122 identifying the electrophysiological signal (and in some instances its location), and associated state change value for identifying electrophysiological signals at locations on the surface of interest similar to an anchor signal. The captured electrophysiological signal can be referred to as an anchor signal and the given location can be referred to as an anchor location. In some examples the anchor signal or the anchor location is identified in response to user input (e.g., at an input device, such as disclosed herein).

The state change detector 118 can identify one or more other locations on the surface of interest showing or exhibiting a similar state changes (e.g., within a given percentage of the given state change, for example, such as about 5 to about 10%, or within a value range, for example, about 2 to about 3) to the anchor location. The state change detector 118 can provide the updated map data 132 with each location on the surface of interest with a similar state change, which can be referred to in some instances as new potential target sites. The state change detector 118 can use a coloring scheme identifying the locations on the surface of interest with the similar state change. For example, the surface of interest at the given location and the locations identified as having the similar state change can have a dark maroon color. This would enable the clinician to modify the treatment based on the updated map data 132 and ablate the new potential target sites.

In some examples, the state change detector 118 can be used to provide the updated map data 132 in response to detecting a feature state change from a first feature state to a second feature state. The state change detector 118 can detect the feature state change in the same or similar manner as disclosed herein. In response to detecting the feature state change, the state change detector 118 can evaluate new compute feature states provided by the feature state calculator 112. If the new computed feature states are about the same (e.g., within a given percentage (e.g., about 5 to about 10%) or value (e.g., about 2 to about 3)) over a given period of time, the state change detector 118 can update the map data 130 to provide the updated map data 132. The map data 130 can be updated based on new captured electrophysiological signals from the surface of interest. In some examples, the state change detector 118 can output an alert (e.g., visual, audible, or a different type) to the clinician to inform the clinician that a new computed feature state has been detected for a period of time and whether the clinician would like to update the map so that electrical activity associated with an arrhythmia can be visualized.

In some examples, one or more different features can be computed for one or more electrophysiological signals. For example, if 200 features are computed for each electrophysiological signal there can be 200 different feature signals (in some instances even more if two different electrophysiological signals are used for computing a respective feature signal). In some examples, the state change detector 118 can provide the feature state change data 122 in response to detecting two or more feature state changes for two or more different features for an electrophysiological signal. The state change detector 118 can provide the feature state change data 122 in response to detecting the two or more feature state changes within a given amount of time of each other (e.g., a defined period of time, or two sampling windows (e.g., the sampling window 110 or the sampling window 114). In some examples, the state change detector 118 provides the feature state change data 122 in response to detecting two or more feature state changes for two or more electrophysiological signals within the given amount of time of each other. Thus, in some examples, the feature state change data 122 is provided if feature state changes are detected for different features across a single channel (e.g., electrophysiological signal) or two or more channels.

The feature state change data 122 provided in response to detecting the two or more feature state changes can characterize a given feature (e.g., CL feature) of features associated with the feature state changes. The given feature can be identified, for example, by identifying which feature of the features associated with the feature state changes has a greatest feature state change, or according to some different criteria, or approach. In some examples, each feature of the two more detected features is identified by the feature state change data 122. As disclosed herein, the feature state change data 122 can be used for providing a disease score and thus weights for generating the disease score can be adjusted based on the features identified by the feature state change data 122. An amount of features (feature amount) for which feature state changes are to be detected for a given channel can be user defined, or set a priori. For example, if the feature amount is set to five this means that five feature changes have to be detected for one or more channels within the given amount of time. Thus, in some examples, a user (e.g., the clinician) can adjust a sensitivity of state change detection by the detector 100.

In some examples, the state change detector 118 can generate a report identifying a number of feature states (for each feature), a frequency at which the feature states change, and relative prevalence/time of each feature state change. The state change detector 118 can relate a collected map point time (for an electrophysiological signal) to which a feature state the arrhythmogenic activity is in and provide data indicating to a user (e.g., that can be rendered on a display) which heart locations need more map points for a given feature state. In some examples, the state change detector 118 can provide data indicating to the user how long to record electrophysiological signals from the heart's surface (e.g., at a given map point) to ensure data is collected across an entire cycle for the given feature state. In some examples, state change detector 118 can implement historical patient matching to others patients with similar feature state values, determine which change types to utilize for the procedure, identify disease score weights that provide best prediction state changes, and successes, for example, to the extent that a p-wave localizer and disease scores assist in target localization. In some examples, a given features state, and change there-to, can be used to simply notify the user when state changes occur, and the user can use that information, for example, to prompt the user to remap heart, instruct the user to continue ablation a certain heart area, go to another area, etc.

In some examples. the feature state can be used to compute a single feature state value that represents a cumulation or summation of multiple feature signals and features. This can be referred to herein as a composite feature state. For example, a feature state calculator can be used to calculate weighting coefficients for each feature state, or variable, in an equation in order to compute a single feature state value that represents an overall electrical activity on the heart surface. For example, features that have highest weights are generally those that represent the whole heart electrical activation patterns and, in some instances, rhythm localizer information.

In some examples, the detector 100 can characterize mapping points for a composite feature state group. For example, the detector 100 can compute or provide a composite state group that can have a mean or median value, and be bounded by minimum and maximum values such that values between the minimum and maximum can include that state group. The max and min group values can be derived by the detector 100 from a variation of a feature signal (e.g., one of the feature signals 106) during that state. For example, a feature signal exhibiting a more consistent point-to-point value variation (e.g., over time) can have a narrower or smaller max and min tolerance around the state value compared to a feature signal exhibiting a more inconsistent point-to-point value variation.

In additional or alternative examples, the detector 100 can search the feature database 124 (or a historical database/library) to find feature signals having similar properties and, if found, uses those thresholds for defining this state group. For example, the detector 100 can find feature signals in the database 124 that are similar across statistics such as a mean, a median, interquartile ratios, a stdev, a variation, a periodicity, an entropy, and other known statistics that capture variation of time series data. In some examples, the detector 100 can calculate state group thresholds from historical database

Historical state group thresholds can be calculated by the detector 100 to values that best cluster or separate the various composite states observed during past case datasets. For example, the detector 100 can utilize hindsight knowledge of all presenting states during a case to cluster the composite states into groups, where states associated with a group are more similar than states in other groups. The thresholds that best separate these state values can be returned and can be used by the detector 100.

In some examples, when the composite state value changes to a new value outside of the group's max and min boundary, the detector 100 can determine this as a candidate new state group. After a given amount of minutes of feature state data, the detector 100 can analyze this data and clusters all presenting states into state groups. For example, the feature detector 100 can derive the thresholds according to the examples disclosed herein (e.g., from steps mentioned above (self-signal properties combined with historical thresholds)), and then apply a bias to the thresholds based upon a patient's baseline state data.

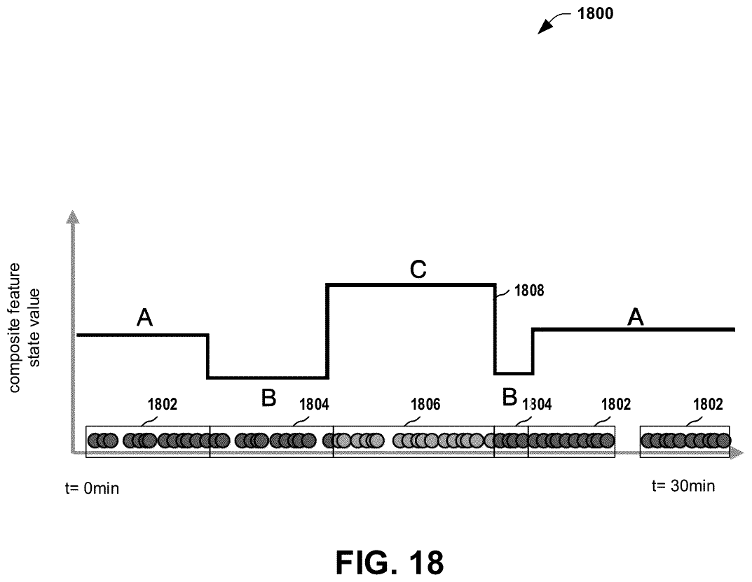

In some examples, the detector 100 can assign each map point (e.g., an electrophysiological signal captured or recorded at a location (e.g., X, Y, Z location) on a surface of interest over a specific time interval) to a state group and calculate a new disease score that's also a function of the present feature state value. For example, the detector 100 can assign each recorded mapping point to the composite state group that was simultaneously present during that mapping point recording. This can be seen in FIG. 18, which is discussed in greater detail below. Each collected point is assigned a score that's a function of, first, the disease score calculated from the electrophysiological signal and, secondly, that disease score in relation to the simultaneous composite arrhythmia state value. For example, each mapping point can be assigned a value computed according to the following equation: y=absolute-disease-score/normalized-composite-state-value (1), wherein y is the value for a respective mapping point, or mapping point value, the absolute-disease-score is the output from a composite disease scoring equation that is a function of all disease feature values.

The composite disease scoring equation can be represented according to the following equation: absolute-disease-score=f((A*F₁+B*F₂+ . . . +B*Fₙ) (2), wherein A, B, are weights, and F is feature values. The normalized-composite-state-value is the composite-state-value that is normalized to the 5% and 95% values, or other proxy max and min values, of all composite state values observed in the matched historical patient group. This primarily highlights mapping signals that have a high absolute disease score relative to a less severe, or lower, feature state value. The user is able to adjust the weighting parameters that govern emphasis on which terms of the equation. Alternatively, these weighting parameters can be auto assigned based upon a machine learning trained model that learns an equation that best predicts if treating a given electrophysiological signal, embodying disease feature values, will elicit a state change alert. This information can be displayed together in a composite map, where the disease values are displayed on a heart model surface. Alternatively or additionally, the user can choose to display one map per state group which will display the disease mapping values observed during that state, or display a combination of state group maps. The user is also able to sort or choose to display disease mapping data from state maps that have the highest severity vs lowest severity, in order to see disease features from map points that were present during specific rhythm or feature states of interest.

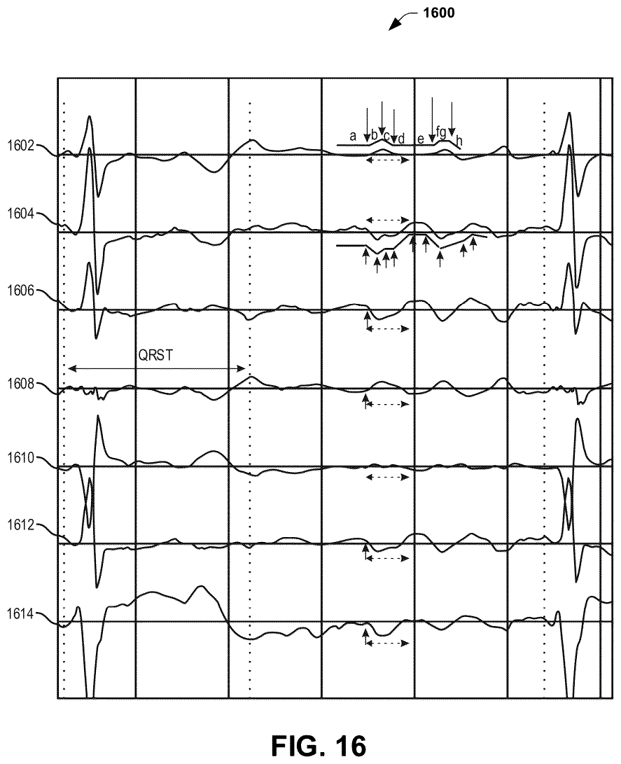

In some examples, the feature detector 100 can detect and characterize active zone instances, such as unipolar p-wave onsets based on relevant electrophysiological data (of the data 104). For example, the feature detector 100 can include a rhythm localizer 138 having a p-wave localizer 140 that can provide localizer data 142 based on the feature states 116 and deflection data provided by the feature signal generator 106. The localizer data 110 can identify location information (e.g., location or nodes (e.g., X, Y, Z locations, or in some instances region or areas on a surface of interest) for potential treatment (e.g., cardiac ablation). While the example of FIG. 1 illustrates the p-wave localizer 140 as part of the rhythm localizer 138, in other instances, it can be implemented as a stand-alone module, or as part of a different model (e.g., of the detector 100, or another system, for example, as disclosed herein). The p-wave localizer 140 can partition (or invoke the feature signal generator 108 to partition) a deflection on an electrophysiological signal (from the electrophysiological data 104) as a contiguous, monotonically increasing or decreasing sequence of voltage measurements. The p-wave localizer 140 (or the feature signal generator 108) can include a deflection algorithm that can be a function of a number of parameters, such as slope, time duration, voltage change, and voltage value. The deflections are shown as reference in A-H in FIG. 16. In a given time interval, the p-wave localizer 140 (or the feature signal generator 108) can search each channel (e.g., the electrophysiological data 104) for a group of deflections that deviate from a nearly flat, or isoelectric, deflection or plurality of sequential deflections. In examples wherein the feature signal generator 108 search channels for the group of deflections, the feature signal generator 108 can provide deflection data identifying the group of deflections to the p-wave localizer 140. These instances (e.g., the group of deflections) can then be compared by the p-wave localizer 140 (or the feature signal generator 108) across unipolar channels (e.g., of corresponding data of the electrophysiological data 104) to evaluate whether other unipolar channels exhibit a significant baseline deviation at nearly the same time as other channels. A localizable instance can be indicated by the p-wave localizer 140 (or the feature signal generator 108) when X #unipolar channels, or Y #unipolar channel groups exhibit this phenomenon at about a same time (e.g., over a given time interval of each channel that overlaps or signals that overlap in time) as other channels. Each of these instances can be stored by the p-wave localizer 140, along with information such as slope, amplitude, deflections, etc. before the instance and the slope, amplitudes, deflections, etc. after the instance (as mentioned above).

Each of these unipolar onset instances in some instances can further be characterized by the p-wave localizer 140 computing and assigning a set of confidence scores per unipolar channel, from which are combined to create a cross channel confidence score per instance. The confidence scores are in part derived from characteristics both prior to and immediately after the onset instance such as, but not limited to, slope of segment, time duration of the segment, voltage values, voltage variation of each signal segment, max voltage change, peak/trough value, and the variation of these values at simultaneous time instances across other unipolar channels. A per channel and cross channel confidence score are each computed by the p-wave localizer 140.

For example, each of these p-wave onset instances can be analyzed by the p-wave localizer 140 to quantify deflection data immediately following the onset instance in order to extract/quantify characteristics of the morphology/shape of the waveform immediately following the onset instance(s). In some examples, an area under a curve is computed by the p-wave localizer 140 from onset time instance through some fixed period of time, or alternatively computed by the p-wave localizer 140 from onset time instances through the time in which a similarly featured deflection or set of deflections exhibiting near-isoelectric properties occurs, or some combination thereof.

In some examples, the p-wave localizer 140 in some instances can calculate additional features in addition to area under the curve such as height, width, AUC, slope etc for each component or a combination of near-similar components, thereby outputting several shape values within a unipolar onset waveform.

In some examples, the deflections, unipolar onset instances, its mentioned underlying information, and the shape feature values of the onset waveform can be computed by the detector 100 per sampling window in order to generate signal features. This can include features such as, but not limited to, a confidence weighted average #of p-wave onset instances, #of p-wave onset instances above a confidence threshold C, the time between instances above a confidence threshold C, variation of the time between instances above a confidence threshold C, the mean/median peak-to-peak amplitude from instance to max or min voltage value, the variation (standard deviation, IQR, clustering of points) of the shape features across an interval, the sequence of deflection components within a wavelet or onset p-wave and variation thereof, etc. These are several examples of many features detector 100 can compute to generate signal features, corresponding feature states, feature state changes, alerts, etc.

In some examples, the rhythm localizer 138, for each cross-channel unipolar onset instance, can assign a percent value to each location or node (e.g., X, Y, Z location), or predefined surface of interest (e.g., heart) region, that reflects a likelihood that the predefined heart region interrupted iso-electricity. For example, the percent value can represent a proxy value for predicting the odds that treating that location can result in a state change. The rhythm localizer 138 can receive calculated features that represent waveform shape. The rhythm localizer 138 inputs waveform shape data for each channel, and outputs percentage likelihood information for each location or node (e.g., on the surface of interest) and predefined region that treating the location will elicit an arrhythmia state change.

In some examples, the detector 100 includes a disease scorer module that inputs and analyzes disease features computed from the electrophysiological signals recorded during mapping, with an associated location or node (e.g., X, Y, Z location on the surface of interest), and for each location or node it outputs a percent likelihood that treatment at that location, or a treatment lesion set inclusive of that location, will elicit a state change alert of high confidence and/or high value. This disease score equation can include a multitude of disease features (as mentioned prior), whose variable coefficients can be trained such that the output is optimized to maximal percent likelihood that treatment of those disease values will elicit a state change.

In some examples, the detector 100 includes a ML model to train the rhythm localizer 138 using state change alert instances that occurred at or nearly immediately thereafter ablation delivery. For example, the detector 100 can identify feature states, particularly those that embody consistent unipolar waveform shape data immediately prior to the state change alert, as the input data. The corresponding output data is the state change alert value that ensued following ablation. The ML model of the detector 100 can be trained by the value of the state change, or the confidence value associated with the state change. These training datasets are each sent into the ML model to more optimally predict, for an input unipolar feature state matrix dataset, which xyz location or predefined region, when ablated, is most likely to immediately elicit a state change alert.

In some examples, the detector 100 includes or utilizes a MLmodel to train the disease scorer. The disease scorer inputs and analyzes the disease features computed from the electrophysiological signals recorded during mapping, with an associated xyz location, and for each xyz location it outputs a percent likelihood that treatment at that location, or a treatment lesion set inclusive of that location, will elicit a state change alert of high confidence and/or high value. A disease score equation of the disease scorer can include a multitude of disease features (as mentioned prior), whose variable coefficients can be trained (eg., by a trainer, which can be part of the detector 118) such that the output is optimized to maximal percent likelihood that treatment of those disease values will elicit a state change.

Alternatively, instead of training these predictive equations using all patient datasets exhibiting an arrhythmia such as Atrial Fibrillation, the detector 100 can train equations that are optimized for a sub-group, or certain phenotype, of the clinical domain of interest. As an example, while a single equation can be trained on all 1,000 datasets of a certain clinical domain of interest, the detector 100 can first cluster these 1,000 datasets into, for example, 10 groups that each have similar properties of disease features and arrhythmia state values. In this example, each group would have an independent predictive equation, which can be trained on data within that group. As such, for the present patient the equation association with the group to which present patient dataset matches would be used for downstream analysis. The newly off-line trained equation can be deployed into software on a periodic basis. Alternatively, the ML model can be run online such that as new data comes directly from the existing case or past case data, the equation is updated after every X number of new training datasets.

In some examples, in addition to independently trained disease score (e.g., training of the disease score equation in embodiments its implement as a ML model) and rhythm localizer 138, these equations can alternatively or additionally be combined into one equation that inputs both disease score matrix values and feature states immediately prior to state change alert and, using an equation embodying these features with corresponding coefficients, predicts the likelihood that treatment at location xyz with corresponding disease score matrix values will result in a state change alert. Alternatively, the disease score and rhythm localizer 138 can each be trained independently first and then can be combined into a master equation. In this example, each equation's contribution to the master target identifier would be governed by a confidence index. The disease score equation confidence index can be calculated (e.g., by the detector 100) by returning a value that represents the extent to which the present patient's baseline feature state values, mapping disease feature values per region, and the disease feature distribution across regions correlate to the historical patient group to which they most closely correlate. The rhythm localizer 138 confidence index can be calculated by returning a value that represents the extent to which the present patient's arrhythmia state has stable localization feature values and its unipolar onset shape value correlates to those in the historical patient group to which they most closely correlate. Accordingly, this computes and displays a target identifier value on the heart surface.

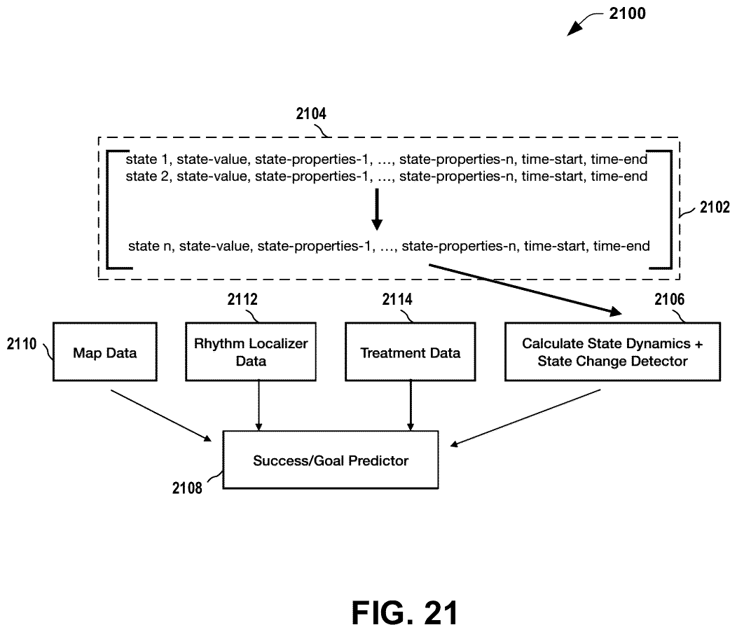

In some examples, the detector 100 and corresponding method disclosed herein can predict, at the current or any prior time during the treatment procedure, a success likelihood by analyzing disease score data from past feature state dynamics. These feature state dynamics, as shown in FIG. 21, represent attributes of each feature state across all signal features representing the electrophysiological signal at fixed spatial locations, which includes feature state values, start and end state times, change between states, sequential state patterns, and other related statistics. Feature state dynamics are calculated by the detector 100 from the feature state attributes such as, but not limited to, feature signal type, state value such as mean median or the alike, state variation such as standard deviation interquartile ratios or the alike, state frequency and periodicity calculations such as time between repeating peaks troughs or similar deflections or the alike, state time start and state time end, and the implicit change values between states for a given signal feature. Feature state dynamics include attributes such as, but not limited to, step change magnitudes in successive feature state changes, frequency of step changes above X magnitude, number of states exhibiting a unique value (straddle tolerance around value) compared to prior state values over a certain time period, location of rhythm localizer variance of a certain time period, among others.

### System for Identifying a Target Site During a Treatment

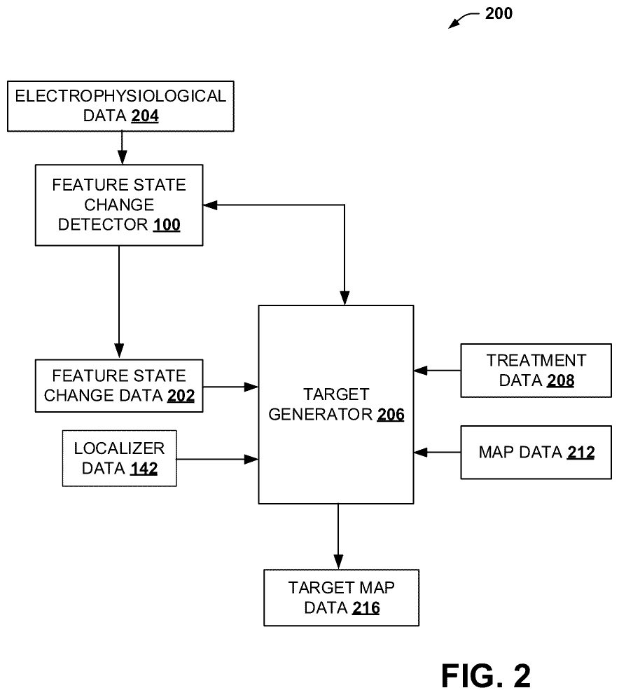

FIG. 2 is a block diagram of a target site identification system 200. The system 200 can include the feature detector 100, as shown in FIG. 1. Thus, reference can be made to the example of FIG. 1 in the example of FIG. 2. The system 200 can be configured to identify one or more target sites within a patient's body based on feature state change data 202. The feature state change data 202 can correspond to the feature state change data 122, as shown in FIG. 1. The feature state change data 202 can be provided based on electrophysiological data 204, which can correspond to the electrophysiological data 104, as shown in FIG. 1. The system 200 includes a target generator 206. The target generator 206 can identify a target site (e.g., for treatment) within a patient's body based on a map generated (by a mapping system 302, as shown in FIG. 3) in response to detecting a feature state change. The feature state change can be associated with a treatment (e.g., an ablation therapy) at a target site on a surface of interest within the patient's body for an electrophysiological event. The target site can be identified prior to the treatment (e.g., during a treatment site identification stage). By way of example, the electrophysiological event can include atrial fibrillation (AF) events, supraventricular tachycardia, premature ventricular contraction (PVC) events, ventricular tachycardia (VT) events, ventricular fibrillation (VF) and/or other types of arrhythmogenic activity/events. In some examples, the target generator 206 can tag each identified target site with a corresponding label as a potential target site for delivery of therapy treatment and/or a type of electrophysiological event.

For example, during ablation, such as cardiac ablation, a potential target site (e.g., an ablation site identified prior to treatment) on a patient's heart (e.g., invasively or non-invasively) can be treated. In the context of cardiac arrhythmia treatment, treating a specific location or circuit in the heart (e.g, the potential target site) can result in changes to an overall arrhythmia circuitry. These changes can often be observed by sensors inside the heart or by ECGs from outside. When treating a critical circuit area, electrical activity can be captured/observed by sensors, such as fixed fiducial sensors. A critical circuit area, in the context of cardiac arrhythmia treatment, can refer to a specific region or pathway in the heart that is responsible for sustaining an arrhythmia. If the treatment is focused on a portion of the critical circuit area, there may be a subtle change observed/detected by the sensors. This subtle change can serve as a cue for a clinician to keep looking and treating nearby areas on the heart because the clinician can be close to a key aspect of that circuit (e.g., a trigger site or a driver region). In some examples, the subtle change can be reflected as a subtle state change, as disclosed herein.

If the treatment is focused on the critical circuit area as a whole (e.g., targeting an entire region of the heart that is responsible for sustaining the arrhythmia), there may be a larger (or major) change observed/detected by the sensors. In some examples, the large change can be reflected as a major state change, as disclosed herein. In some examples a rhythm localizer is used to determine a location of the critical circuit area, and thus the location of the arrhythmia in the heart. In some examples, the rhythm localizer is a fixed fiducial sensor that can be placed on the surface of the heart or in a blood vessel near the heart to record electrical activity of the heart during an arrhythmia. Changes in the electrical signals recorded by the rhythm localizer can indicate whether the treatment is successful, or if further treatment is needed. For example, if the electrical activity captured or recorded by the rhythm localizer changes, it may indicate to the clinician that the clinician should move on to a different location for further treatment. However, if the electrical activity captured or recorded by the rhythm localizer does not change (or by a given amount or percentage), it may mean that there is more to be treated in that area or that the clinician should continue to treat nearby.

In some examples, treating a specific location successfully can unmask a previously dormant or less obvious arrhythmia driver. A detected state change associated with a therapy applied at a target site can be indicative that the clinician is in proximity, near, touching and/or adjacent a diseased circuit and this detected state change can be used to identify other locations on the surface of interest exhibiting a similar state change change. As such, treatment at a given location of the heart can reveal underlying factors that were contributing to the arrhythmia but were not initially apparent. This can help clinicians tailor their treatment approach and achieve better outcomes for their patients.

Signal patterns are not uniform across patient's hearts and can vary (in some instances significantly), making it difficult to determine a most effective treatment approach for each patient. For example, an AF driver's signal disease signature is not uniform across patients and as such electrical signal patterns associated with AF in a patient's heart vary between patients. According to the examples disclosed herein, the disease features of electrophysiological signals (e.g., the electrophysiological data 204) are evaluated at previously recorded locations that caused state changes during treatment, such as ablation. These underlying signals and their corresponding disease features are then analyzed to identify any common threads or similar disease features or characteristics and also exist across the other electrophysiological signals and their disease features. This analysis provides an anchor signal or anchor feature values that serve as a reference point for further analysis, as disclosed herein. In some examples, if no convergent themes or common disease threads are identified across signals associated with a treated location that elicited a state change, then this feature can be disabled, or left blank. In other examples, a single anchor signal can be used and its disease features to identify and highlight these other locations. In some examples, a visualization tool can be provided to show a user (e.g., the clinician) which mapping signals (or locations on the surface of interest) have most similar disease feature values to the anchor signal or manually chosen anchor feature values. This information can help the clinician determine a most effective treatment approach for each patient based on their unique AF signal disease features, and which disease feature values, when treated, may be most likely to elicit future state changes for that patient that progress towards procedure success.

In some examples, the anchor signal and anchor feature values can be calculated or extracted from data within a historical patient sub-group which has some combination of similar baseline disease feature values, similar feature state dynamics, and/or an anchor signal or anchor disease features that matches closely with that of the existing event in the procedure.

In some examples, arrhythmogenic activity at other locations that exhibit state values that were previously unseen in prior states can be detected and thus identified (e.g., unmasked). By detecting feature state changes, the target generator 206 and thus the system 200 can detect arrhythmogenic activity at the other location caused by ablation at a prior identified target site, thereby allowing or enabling a clinician to modify or adjust on an on-going treatment in real-time to improve an efficacy of the treatment. Accordingly, a detected feature state change associated with a given location (target site) can be used to identify arrhythmogenic activity at another location on a surface of the patient's heart.

For example, the target generator 206 can receive the feature state change data 202, treatment data 208, and map data 212 for identifying a potential target site on the surface of interest within the patient's body that was not identified prior to treatment. Target sites identified by the target generator 206 can be referred to herein as potential target sites and target sites identified before the treatment can be referred to as prior target sites. The time at which the treatment (e.g., ablation) was applied to the surface of interest within the patient's body can be provided as part of the treatment data 208 and can be referred to as a treatment time. In some examples, the treatment data 208 can identify a location at which ablation was applied.

The map data 212 can include a map that can represent reconstructed electrophysiological signals (of the electrophysiological data 204) spatially and temporally on the surface of interest within the patient's body. The map can be generated and thus updated based on updated electrophysiological signals from sensors within and/or on the patient's body. The map can identify (e.g., using a graphical element, or some other type of indicia) one or more prior identified target sites. The map (or other related data) can also identify a location on the surface of interest for each reconstructed electrophysiological signal.

In some examples, the target generator 206 can create a region on the surface of interest within the patient's body based on region criteria. The region criteria can identify a boundary of the region. Thus, in some examples, the region is a complete surface of the interest or a portion thereof (e.g., of an anatomical structure) within the patient's body. The target generator 206 can identify electrophysiological signals within the region, which can be referred to as region electrophysiological signals. Each electrophysiological signal reconstructed on the surface of interest can have an associated surface position. The target generator 206 can identify a mapping time interval for the region electrophysiological signals. The mapping time interval can be provided to the feature detector 100 to receive the feature signals.

For example, the feature detector 100 can provide the features signals having a feature timing interval that overlaps with the mapping time interval and these feature signals (or portions thereof) can be referred to as region feature signals. In some examples, the target generator 206 can identify respective portions of the feature signals generated by the feature detector 100 using the mapping time interval and the feature timing interval. In other examples, the feature detector 100 can identify the respective portions of the feature signals and provide respective feature signal portions to the target generator 206 based on the mapping and featuring timing intervals. As such, the target generator 206 can request the feature signals based on the mapping time interval In some examples, the feature state change data 202 can indicate that feature state changes have occurred for sub-region feature signals in response to the treatment of the prior target site. A respective sub-region feature signal can be identified and referred to as an anchor signal. In some examples, the respective sub-region feature signal can be associated with the prior target site.

The target generator 206 can identify one or more sub-region feature signals (and thus corresponding locations on the surface of interest) based on feature state change data 202. For example, the target generator 206 can identify the one or more sub-region feature signals exhibiting a similar state changes (e.g., within a given percentage of the given state change, for example, such as about 5 to about 10%, or within a value range, for example, about 2 to about 3). In some examples, if a state change occurs for a feature signal of a number of feature signals for a location on the surface of interest, the target generator 206 can evaluate or look at all state changes for remaining feature signals at the location to identify the one or more sub-region feature signals of the region feature signals. For example, the target generator 206 can identify the one or more sub-region feature signals based on a given number of feature values being greater or equal to a given value specified by the feature state change data 202 for the respective sub-region feature signal.

The target generator 206 can identify one or more sub-region feature signals (and thus corresponding locations on the surface of interest) based on feature state change data 202. For example, the target generator 206 can identify the one or more sub-region feature signals exhibiting a similar state changes (e.g., within a given percentage of the given state change, for example, such as about 5 to about 10%, or within a value range, for example, about 2 to about 3). In some examples, if a state change occurs for a feature signal of a number of feature signals for a location on the surface of interest, the target generator 206 can evaluate or look at all state changes for remaining feature signals at the location to identify the one or more sub-region feature signals of the region feature signals. For example, the target generator 206 can identify the one or more sub-region feature signals based on a given number of feature values being greater or equal to a given value specified by the feature state change data 202 for the respective sub-region feature signal.

The target generator 206 can use the identified one or more sub-region feature signals to identify one or more sub-region electrophysiological signals from which the one or more sub-region feature signals have been provided (or generated). The target generator 206 can output target map data 216 identifying one or more locations on the surface of interest based on the identified one or more sub-region electrophysiological signals (corresponding to reconstructed electrophysiological signals) on the surface of interest. As disclosed herein, the map data 212 or other data can be used to provide each location for a respective reconstructed electrophysiological signal on the surface of interest. Each location identified by the target map data 216 can be potential target sites for treatment (e.g., ablation). In some examples, the target generator 206 updates the map data 212 to provide the target map data 216, which, in some instances, can correspond to the updated map data 132, as shown in FIG. 1. Thus, in some examples, the target generator 206 can output the target map data 216 with the map with each potential target site graphically differentiated from prior target sites (e.g., identified prior to treatment). In some examples, the target map data 216 can be provided to the mapping system to update the map data 212 to include each potential target site identified by the target generator 206. In other examples, the target map data 216 can be provided with the map with each potential target site identified therein. In some examples, the target map data 216 can be provided on the localizer data 142, as shown in FIG. 1. As disclosed herein, the p-wave localizer can provide the localizer data identifying potential sites (or locations), or areas/regions that can be flagged as potential target sites for treatment (e.g., ablation). In some examples, the target map data 216 is provided based on the feature state change data 202, the treatment data 208, the localizer data 142, and/or the map data 212.

In some examples, the target map data 216 can be provided on the localizer data 142, as shown in FIG. 1. As disclosed herein, the p-wave localizer can provide the localizer data identifying potential sites (or locations), or areas/regions that can be flagged as potential target sites for treatment (e.g., ablation). In some examples, the target map data 216 is provided based on the feature state change data 202, the treatment data 208, the localizer data 142, and/or the map data 212. In some examples, the target generator 206 can provide the target map data 216 identifying each location on the surface of interest with a similar state change as the respective sub-region feature signal. In some examples, the target generator 206 can assign a similar color to each identified location on the surface of interest with the similar state change as the respective sub-region feature signal. For example, a given location associated with the respective sub-region feature signal and locations identified as having the similar state change as the respective sub-region feature signal can have a dark maroon color. This would enable the clinician to modify the treatment based on the target map data 216 and treat the new potential target sites. In some examples, new received electrophysiological data can be processed to label (e.g., by a color, or flag in a different way) locations on the surface of interest having a similar state change as the respective sub-region feature signal. In other examples, the newly received electrophysiological data is processed to identify a new anchor signal according to the examples disclosed herein, and the process is repeated to identify signals with similar state changes as the new anchor signal.

### Electrocardiographic Mapping and Target Site Identification System

FIG. 3 is a block diagram of an electrocardiographic mapping and target site identification system 300. The system 300 includes a mapping system 302 for generating one or more maps, for example, as described herein. The mapping system 302 can be implemented as hardware (e.g., circuit and/or devices), software (e.g., a non-transitory medium having machine-readable instructions), or a combination of hardware and software. In some examples, the mapping system 302 can combine electrophysiological data 304 with geometry data 306 to generate map data 308. The electrophysiological data 304 can correspond to the electrophysiological data 104, as shown in FIG. 1, or the electrophysiological data 204, as shown in FIG. 2. Thus, reference can be made to the examples of FIGS. 1-2 in the example of FIG. 3. The map data 308 can include a map that can represent reconstructed electrophysiological signals spatially and temporally on a surface of interest within a body of a patient. In some instances, the map data 308 can correspond to the map data 130, as shown in FIG. 1, or the map data 212, as shown in FIG. 2. In some examples, the map of the map data 308 includes an anatomical structure within the patient's body without the electrophysiological signals reconstructed therein.

In some examples, the mapping system 302 can receive the electrophysiological data 304 representing electrophysiological signals measured from the outer surface or within the patient's body. In some examples, the mapping system 302 can generate the map data 308 based on target map data 310 generated by the target generator 206. The target generator 206 can generate the target map data 310 in a same or similar manner as disclosed herein. Thus, in some instances, the target map data 310 can correspond to the target map data 216, as shown in FIG. 2.

Continuing with the example of FIG. 3, the electrophysiological data 304 can be stored with the geometry data 306 in memory (e.g., one or more non-transitory computer readable media, for example, as disclosed herein) as electroanatomic data that describes the electrical activity at a plurality of locations (e.g., nodes) across the surface of interest (e.g., in three-dimensional space). The locations (or anatomical locations) can be represented as nodes distributed (e.g., an even distribution) over the surface of interest, represented by the geometry data 306. The surface of interest can be a surface of an anatomical structure, such as a tissue of a patient (e.g., human or other animal). In some examples, the patient tissue can be cardiac tissue, such that the surface of interest corresponds to an epicardial surface, an endocardial surface, or another cardiac envelope. The surface of interest can be patient-specific (e.g., based on imaging data for the patient), it can be a generic model of the surface or it can be a hybrid version of a model that is customized based on patient-specific data (e.g., imaging data, patient measurements, reconstructed data, and/or the like).

As disclosed herein, the surface of interest can be defined by the geometry data 306 that is stored in the memory. In some examples, the geometry data 306 can represent a two-dimensional or a three-dimensional surface for the patient. For example, a measurement surface can include a body surface (e.g., an outer surface of the thorax or portion thereof) where sensors are positioned to measure electrical activity. In other examples, the surface of interest can be a surface of internal tissue or a computed envelope having a prescribed position relative to certain internal tissue. Depending on the surface of interest for which the electrophysiological data 304 has been provided, the geometry data 306 can correspond to actual patient anatomical geometry, a preprogrammed generic model, or a hybrid thereof (e.g., a model that is modified based on patient anatomy).

In some examples, the geometry data 306 can be derived from image data 314 generated by an imaging modality. Examples of imaging modalities include ultrasound, computed tomography (CT), 3D Rotational angiography (3DRA), magnetic resonance imaging (MRI), x-ray, positron emission tomography (PET), fluoroscopy, and the like. Such imaging can be performed separately (e.g., before) the measurements utilized to generate electrical data (e.g., the electrophysiological data 104, as shown in FIG. 1, the electrophysiological data 204, as shown in FIG. 2, or the electrophysiological data 304, as shown in FIG. 3). Another example of a modality used to generate the 3D heart surface is by localizing the catheter, via magnetic, impedance based, or the link, within a coordinate system and rendering heart surface as the outermost aspect of this volume, once the clinician completes the step. By way of further example, the geometry data 306 can be acquired using nearly any imaging modality based on which a corresponding representation of a geometrical surface can be constructed, such as disclosed herein. Such imaging may be performed concurrently with the recording of the electrophysiological signals that are utilized to generate the electrophysiological data 304 or the imaging can be performed separately (e.g., before or after the electrophysiological data 304 has been acquired).

In some examples, the mapping system 302 is configured to derive the geometry data 306 from the image data 314. As disclosed herein, the surface of interest can correspond to a three-dimensional surface geometry corresponding to a patient's heart, whose surface can be epicardial and/or endocardial. Alternatively or additionally, the cardiac envelope can correspond to a geometric surface that resides between the epicardial surface of a patient's heart and the surface of the patient's body where a sensor array has been positioned. Additionally, the geometry data 306 that is utilized by the mapping system 302 can correspond to actual patient anatomical geometry, a preprogrammed generic model, or a combination thereof (e.g., a model that is modified based on patient anatomy).

As an example, the geometry data 306 may be in the form of a graphical representation of the patient's torso, such as the image data 314 acquired for the patient. Image processing, which in some examples, can be implemented by the mapping system 302, can include extraction and segmentation of anatomical features, including one or more organs and other structures, from a digital image set corresponding to the image data 314. Additionally, a location for each of the sensors in the sensor array can be included in the geometry data 306, such as by acquiring the image while the sensors are disposed on the patient and identifying the electrode locations in a coordinate system through appropriate extraction and segmentation. Other non-imaging based techniques can also be utilized to obtain the position of the sensors in the sensor array, such as a digitizer or manual measurements.

In some examples, the geometry data 306 can correspond to a mathematical model, which can be a generic model or a model that has been constructed based on the image data 314 for the patient. Appropriate anatomical or other landmarks, including locations for the sensors in the sensor array, can be identified in the geometry data 306 to facilitate registration of the electrophysiological data 304. The identification of such landmarks can be done manually (e.g., by a person via image editing software) or automatically (e.g., via image processing techniques), in some examples, can be implemented by the mapping system 302.

In some examples, the mapping system 302 can include a reconstruction engine 316. The reconstruction engine 316 can compute reconstruct electrophysiological signals on the surface of interest within the body of the patient based on the electrophysiological data 304 and the geometry data 306. For example, the reconstruction engine 316 can combine the electrophysiological data 304 and the geometry data 306. The reconstruction engine 316 can reconstruct a body surface electrical activity measured via the sensors (e.g., the sensor array) on a body of the patient onto a multitude of locations on a cardiac envelope corresponding to a sub-region of the heart for the electrophysiological event. In an example, the reconstruction engine 316 can use user input data 318 that identifies one or more regions on the anatomical structure that have to be ablated for generating mapping data/information. Thus, the reconstruction engine 316 can use prior ablation information for the anatomical structure of the patient.

As a further example, the mapping system 302 can compute a map (e.g., one or more cardiac or disease maps) based on the reconstructed electrophysiological signals on the surface of interest within the body. A disease map can refer to a map of a surface of interest with target sites (e.g., new target sites identified according to the examples disclosed herein, in some instances prior target sites identified prior to treatment) identified on the surface of interest, and/or having disease scores for locations on the surface of interest. To compute the map, the mapping system 302 can include a map generator 320. The map generator 320 can generate the map representing the reconstructed electrophysiological signals on the surface of interest during the respective time interval. Thus, the map generator 320 can reconstruct the electrophysiological signals on the surface of interest rendered on an anatomical model of the surface of interest. The map generator 320 further may compute or derive one or more maps from the reconstructed electrophysiological signals to characterize features of the reconstructed signals across the surface of interest during the respective time interval. The mapping system 302 can provide the map (or maps) as the map data 308, as shown in FIG. 3.

In some examples, the map data 308 can be provided to (or accessed by) the target generator 206. The target generator 206 can identify each target site for the treatment within the body of the patient based on the map data 308 in a same or similar manner as disclosed herein. The target site identified by the target generator 206 can specify a location for delivery of a therapy to treat the patient. In some examples, the map generator 320 can provide the map data 308 based on prior site data 324. The prior site data 324 can be generated prior to ablation therapy, can identify a number of target sites at which the surface of interest is to be ablated, and thus can be referred to as prior target sites. The map generator 320 can provide the map data 308 with the prior target sites identified on the surface of interest based on the prior site data 324. In some examples, the target generator 206 can update the map data 308 to provide the target map data 310 with each new target site graphically differentiated from the prior target sites. In some examples, the mapping system 302 includes functionality of the detector 100 so that the mapping system 302 can provide the updated map data 132, as shown in FIG. 1. Thus, in some examples, the target map data 310 can correspond to the updated map data 132. In some examples, the target generator 206 can provide a map with each new target site identified therein as the target map data 310. The map with each target site (corresponding to the target map data 310) and the map with each prior target site (corresponding to the map data 308) can be rendered on a display (e.g., next to each other) to facilitate the on-going treatment, in some instances.

In some examples, the surface of interest (e.g., a geometric surface) can be represented as a mesh that includes a plurality of nodes interconnected by edges to define the mesh. The target generator 206 thus can generate the target map data 310 identifying the target site as one or more nodes of the plurality of nodes of the mesh based on the map data 308. In some examples, the surface of interest can be represented by a mesh that includes a collection of vertices that define a plurality of polygons forming the surface of interest. Each polygon can include three or more vertices, such that adjacent vertices define edges that surround a face of each respective polygon. The target generator 206 can generate the target map data 310 identifying the target site as a region (or as a centroid of the region) enclosed by one or more polygons of the plurality of polygons. As a further example, the map data 308 and/or the target map data 310 can be provided to a visualization engine, which can be configured to render map data 308 and/or the target map data 310 on a display to provide a visualization of each target site and/or prior target site, and in some instances with tagging information identify each type of target site.

In some examples, the target generator 206 can denote each target site on the surface of interest to differentiate the target site from surrounding regions on the surface of interest and/or the prior target sites on the surface of interest. For example, each target site identified by the target generator 206 and/or the prior target sites can be denoted with a corresponding color from a color scale or other scale to distinguish the target site on the map. Accordingly, the target generator 206 can provide the target map data 310 that includes the map with each target site graphically differentiated (e.g., from the prior target sites).

### Diagnostics and/or Patient Treatment System

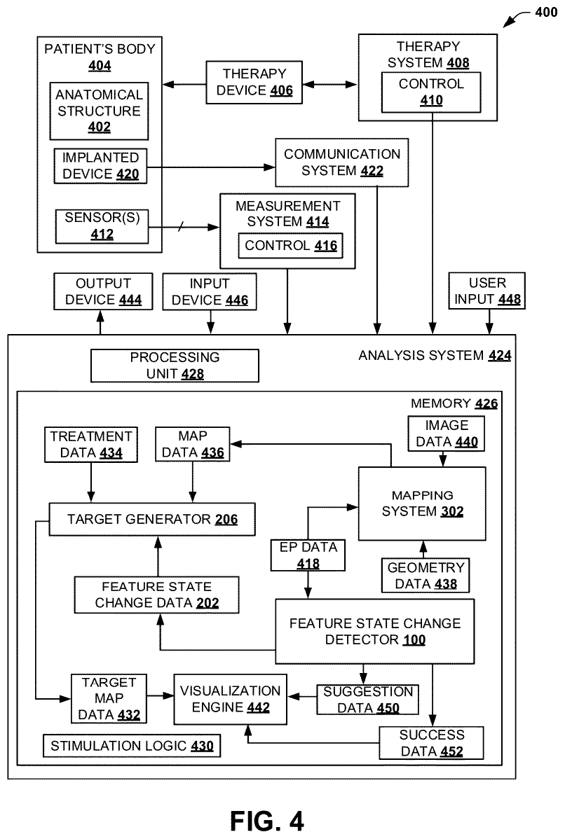

FIG. 4 depicts an example of a system 400 that can be utilized for performing diagnostics and/or treatment of a patient, such as non-invasive or invasive ablation. In some examples, the system 400 can be implemented to generate corresponding maps (e.g., cardiac maps) for an anatomical structure 402 within a patient's body 404 in real time as part of a diagnostic procedure (e.g., an electrophysiology study) to help assess electrical activity and identify one or more prior target sites prior to ablation. Additionally, or alternatively, the system 400 can be utilized as part of a treatment procedure, such as to help a physician to identify other targets sites during ablation, and in some instances, determine parameters for delivering a therapy to the patient (e.g., and amount, and type of therapy) based the other target sites identified according to the systems and methods disclosed herein.

For example, the patient's body 404 can be positioned relative to a therapy delivery device 406. In one example, the therapy delivery device 406 is a catheter configured for delivering ablation treatment to the anatomical structure 402. In some examples, the therapy delivery device 406 is a linear accelerator. The therapy delivery device 406 can be configured to deliver radiotherapy using gamma rays or x-rays to each target site. As a further example, a therapy system 408 can be located external to the patient's body 404 and be configured to control therapy that is being delivered by the therapy delivery device 406 non-invasively or invasively, as disclosed herein.

For instance, the therapy system 408 includes controls (e.g., hardware and/or software) 410 that can be configured to communicate (e.g., supply) electrical signals via a conductive link electrically connected between the therapy delivery device and the therapy system 408. The controls 410 can control parameters of the therapy delivery device 406 (e.g., an amount of energy) for delivering the therapy (e.g., ablation) to the identified target sites. The controls 410 can set therapy parameters and apply stimulation based on automatic, manual (e.g., user input), or a combination of automatic and manual (e.g., semiautomatic) controls. The position of the therapy delivery device 406 relative to the anatomical structure can be determined and/or tracked intraoperatively via a medical imaging modality (e.g., fluoroscopy, x-ray, ultrasound, and the like), direct vision, or the like.

Before, during and/or after delivering the therapy via the therapy system 408, another system or subsystem can be utilized to acquire electrophysiology information for the patient. In the example of FIG. 4, a sensor array 412 includes electrodes that can be utilized for measuring patient electrophysiological signals. In some examples, the sensor array 412 includes one or more electrodes positioned within the patient's body on a lead or a basket catheter. In other examples, the sensor array 412 can correspond to a high-density arrangement of body surface sensors (e.g., greater than approximately 50-200 electrodes) that are distributed over a portion of the patient's torso for measuring electrical activity associated with the anatomical structure (e.g., as part of an electrocardiographic mapping procedure), such as described above. Other arrangements and numbers of sensing electrodes can be used as the sensor array 412.

As an example, the array can be a reduced set of electrodes (e.g., a 12-Lead ECG), which does not cover the patient's entire torso and is designed for measuring electrical activity for a particular purpose (e.g., an array of electrodes specially designed for analyzing AF and/or VF events) and/or for monitoring a predetermined spatial region of the heart. In some examples, one or more sensors may also be located on the therapy delivery device 406. Such sensors can also be utilized to help localize the therapy delivery device 406 within the anatomical structure 402, which can be registered into an image or map that is generated by the system 400.

The sensor array 412 can be configured to provide the sensed electrical information to a corresponding measurement system 414. The measurement system 414 can include appropriate controls and signal processing circuitry and control 416 for providing corresponding electrophysiological data 418 (shown as “EP DATA” in FIG. 4) describing electrical activity measured by the sensors in the sensor array 412. The electrophysiological data 418 can include analog and/or digital information (e.g., corresponding to electrophysiological data 104, as shown in FIG. 1, the electrophysiological data 204, as shown in FIG. 2, or the electrophysiological data 304, as shown in FIG. 3). A control 416 of the measurement system 414 can be configured to control the data acquisition process (e.g., sample rate, line filtering) for measuring electrical activity and providing the electrophysiological data 418.

In some examples, the control 416 can control the acquisition of the electrophysiological data 418 separately from the therapy system operation, such as in response to a user input. In other examples, the electrophysiological data 418 can be acquired concurrently with and in synchronization with delivering therapy by the therapy system, such as to detect electrical activity of the anatomical structure 402 that occurs in response to applying a given therapy (e.g., according to therapy parameters). For instance, appropriate time stamps can be utilized for indexing the temporal relationship between the electrophysiological data 418 and therapy parameters used to deliver therapy to facilitate the evaluation and analysis thereof.

In some examples, a device (e.g., an implantable medical device) 420 is implanted within the patient's body 404. The implanted device 420 can communicate with a communication system 422. The implanted device 420 can be configured to communicate wirelessly with an analysis system 424 via the communication system 422. By way of example, the communication system 422 is a wireless system, such as a Wi-Fi system (e.g., Wi-Fi network). In some examples, the communication system 422 can be configured to allow for communication of data between the implanted device 420 and the analysis system 424 according to a wireless technology standard, such as near field communication (NFC), Bluetooth or Wi-Fi standard.

The analysis system 424 can be implemented as including a computer, such as a laptop computer, a desktop computer, a server, a tablet computer, a workstation, or the like. In some examples, the analysis system is implemented in a cloud-computing environment or platform. The analysis system 424 can include memory 426 for storing data and machine-readable instructions. The memory 426 can be implemented, for example, as a non-transitory computer storage medium, such as volatile memory (e.g., random access memory), non-volatile memory (e.g., a hard disk drive, a solid-state drive, flash memory, or the like) or a combination thereof. The instructions can be programmed to perform one or more methods, such as disclosed herein with respect to the example of FIGS. 1-3. The analysis system 424 can include a processing unit 428 to access the memory 426 and execute the machine-readable instructions stored in the memory 426. The processing unit 428 could be implemented, for example, as one or more processor cores. In the present examples, although the components of the analysis system 424 are illustrated as being implemented on the same system, in other examples, the different components could be distributed across different systems (in some instances in the cloud computing environment) and communicate, for example, over a network.

In some examples, the analysis system 424 employs stimulation logic 430 to provide pacing parameters (e.g., pacing protocols) or other instructions to control the implanted device 420 via the communication system 422. In some examples, the communication system 422 communicates pacing parameters or other instructions through a wireless communications link. The link may be a direct link or an indirect link through a programmer device. The implanted device 420 can be configured to induce an electrophysiological event at the anatomical structure 402 based on the pacing parameters. Thus, in some examples, the implanted device 420 can be configured to induce an AF event at the anatomical structure 402. In additional examples, the implanted device 420 can be configured to terminate the induced electrophysiological event at the anatomical structure 402 based on the pacing parameters. In some examples, the sensor array 412 can be configured to sense electrophysiological signals corresponding to the electrical activity at the anatomical structure 402 in response to the implanted device 420 inducing the electrophysiological event. In some examples, the electrophysiological event is not induced by the implanted device 420 and the patient is monitored via the sensor array 412 to capture the electrophysiological signals from the patient's body 404 (or within the patient's body in other implementations using the catheter) for a natural occurrence of the electrophysiological event as well as other physiological conditions.

In some examples, the electrophysiological data 418 can be stored in the memory 426. The memory 426 includes a target generator 206 that can output target map data 432 based on treatment data 434 (which can correspond to the treatment data 208, as shown in FIG. 2), and/or map data 436 in a same or similar manner as disclosed herein. In some examples, the map data 436 can correspond to the map data 130, as shown in FIG. 1, the map data 212, as shown in FIG. 2, or the map data 308, as shown in FIG. 3. The map data 436 can be provided by the mapping system 302 in a same or similar manner as disclosed herein. In some examples, the feature detector 100, the target generator 206, and/or the mapping system 302 can provide updated map data, such as the updated map data 132, according to the examples disclosed herein.

In some examples, the mapping system 302 can reconstruct electrophysiological signals on a surface of interest of the anatomical structure 402 within the patient's body 404 based on non-invasively or invasively measured electrophysiological signals and geometry data 438. The geometry data 438 can correspond to the geometry data 306, as shown in FIG. 3. Thus, the geometry data 438 can be a three-dimensional anatomical model of the anatomical structure 402 and the body surface where the electrophysiological signals are non-invasively or invasively measured. In some examples, the geometry data 438 can be derived from image data 440. The image data 440 can be provided by an imaging modality and can include one or more images of the anatomical structure 402. In some examples, the image data 440 corresponds to the image data 314, as shown in FIG. 3.

In some examples, the mapping system 302 can compute an inverse solution to reconstruct electrophysiological signals on the surface of interest of the anatomical model of the anatomical structure 402 within the patient's body 404 based on the electrophysiological data 418, and the geometry data 438. The mapping system 302 can generate a map representing the reconstructed electrophysiological signals on the surface of interest, for example before and/or during therapy.

In some examples, the map data 436 is provided to the target generator 206 to provide a map with target sites identified therein according to the examples disclosed herein as the target map data 432. In some implementations, the map can also include the prior target sites identified before the treatment. The prior target sites and the target sites identified according to the examples herein can be graphically differentiated on the map. In some examples, the map only identifies each target site and the prior target sites can be removed or eliminated from the map data 436 used to generate the target map data 432. In further or additional examples, the map includes disease scores for locations on the surface of interest computed according to the examples disclosed herein. Thus, in some examples, the detector 100 can provide disease score data characterizing the disease scores for the locations on the surface of interest, which the system 302 can use to provide the map data 436.

In some examples, the target generator 206 can communicate the target map data 432 to a visualization engine 442. In some examples, the visualization engine 442 can receive the map data 436. The visualization engine 442 can render the target map data 432 and/or the map data 436 on an output device 444 (e.g., a display). In some examples, a user can employ an input device 446 (e.g., a keyboard, a mouse, and the like) to flag each target site (e.g., for evaluation after ablation) based on the visualization rendered on the output device 444. In some examples, the input device 446 can be used to provide user input data 448. The user input data 448 can identify each selected target site. In some examples, the user input data 448 can include the user input data 318, as shown in FIG. 3. In some examples, the visualization engine 442 can render a first map based on the target map data 432 and a second map based on the map data 436 on the output device 444. In some instances, the visualization engine 442 can merge the map data 436 and the target map data 432 to overlay the first and second maps as a merged map on the output device 444.

For example, during cardiac ablation, a user can employ the therapy delivery device 406 to ablate a prior target site on the surface of interest of the anatomical structure 402. Feature state change data associated with the ablation of the prior target site can be used to identify arrhythmogenic activity at another (nearby or neighboring) location or region on the surface of interest of the anatomical structure 402. The target generator 206 can identify the other location or region on the anatomical structure 402 (through feature state changes, as disclosed herein) to provide the target map data 432. The target generator 206 can provide a map with each location identified therein as potential target sites for ablation during the treatment, and some instances, for another treatment.

In some examples, the feature detector 100 can provide treatment suggestion data 450 and/or success data 452. The treatment suggestion data and the treatment success data can correspond to the treatment suggestion data 128 and treatment success data 126, respectively, as shown in FIG. 1. The treatment suggestion data 450 and the treatment success data 452 can be generated by the feature detector 100 in a same or similar manner as disclosed herein. Each of the treatment suggestion data 450 and the treatment success data 452 can be provided to the visualization engine 442 for rendering on the output device 444 during the treatment. As disclosed herein, the treatment suggestion data 450 and the treatment success data 452 can be generated by the feature detector 100 in response to a feature state change and drive (e.g., influence) the (on-going) treatment, such as by indicating how successful the treatment is proceeding, or provide a suggestion (e.g., using visual cues on the display, or in other instances, an audible sound) on whether the clinician should continue the treatment.

### Electrophysiological Signal Plots

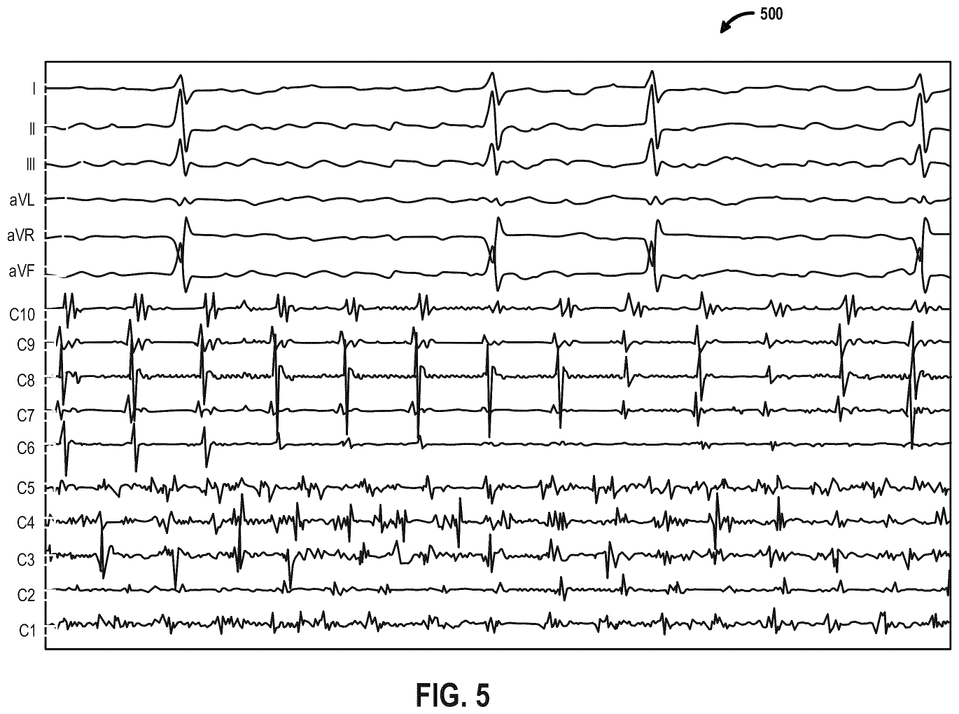

FIG. 5 is an example of a plot 500 of electrophysiological signals (identified as I, II, III, aVL, AVR, aVF, VI, CS_1-2, CS_3-4, CS_5-6, CS_7-8, and CS_9-10) over a period of time e.g that can be provided as (or as part of) the electrophysiological data 104, as shown in FIG. 1, the electrophysiological data 304, as shown in FIG. 3, or the electrophysiological data 418, as shown in FIG. 4. Thus, reference can be made to the examples of FIGS. 1-4 in the example of FIG. 5. The electrophysiological signals can be measured from a human or patient (e.g., via sensors distributed across a surface of a patient's body and/or using a catheter), as disclosed herein. In some examples, a set of electrophysiological signals can be provided as electrophysiological data to feature detector 100 for analysis and/or processing according to the examples disclosed herein. While in the example of FIG. 5 a set of electrophysiological signals are provided to the feature detector 100, in other examples, all or a different number of electrophysiological signals can be provided to the feature detector 100 for analysis and/or processing according to the examples disclosed herein.

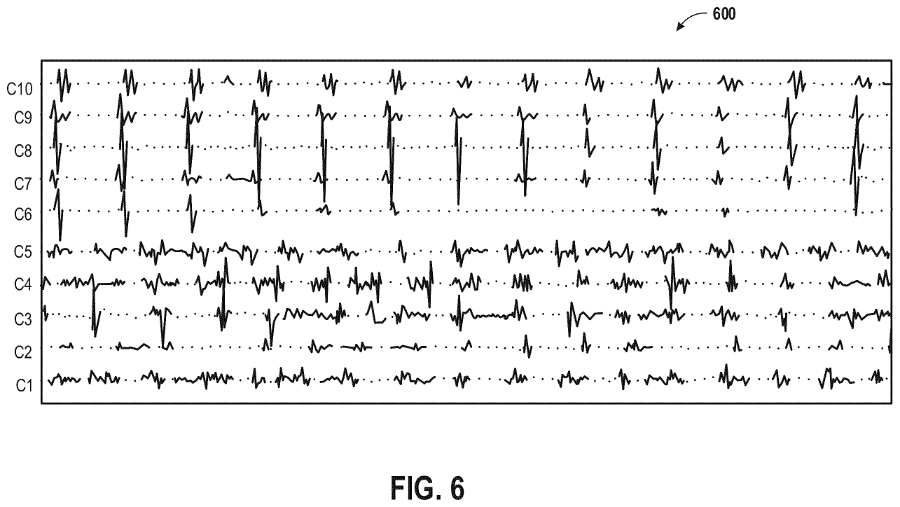

FIG. 6 is an example of a plot 600 of the electrophysiological signals from the example of FIG. 5 in which active and non-active portions have been identified, or electrophysiological signal segments have been identified for feature signal generation. Thus, reference can be made to the examples of FIGS. 1-5 in the example of FIG. 6. As disclosed herein, the feature detector 100 can process the set of electrophysiological signals to identify active portions (e.g., the electrophysiological signal segments) from each electrophysiological signal segment. In the example of FIG. 6, the active portions of the electrophysiological signals are shown with a dashed-line while the non active portions are shown with a solid-line. The active portions of the electrophysiological signals can be processed by the feature signal generator 108 of the detector 100 for feature signal generation (e.g., providing the feature signals 106, as shown in FIG. 1).

### Disease Maps

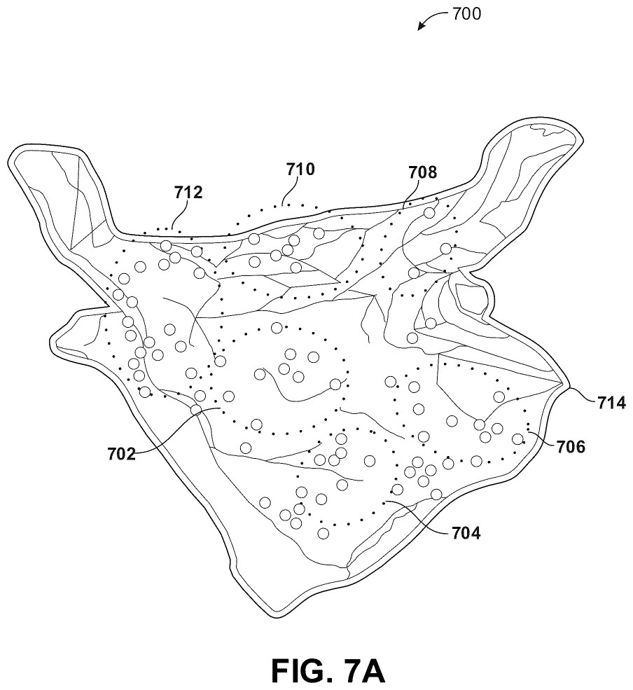

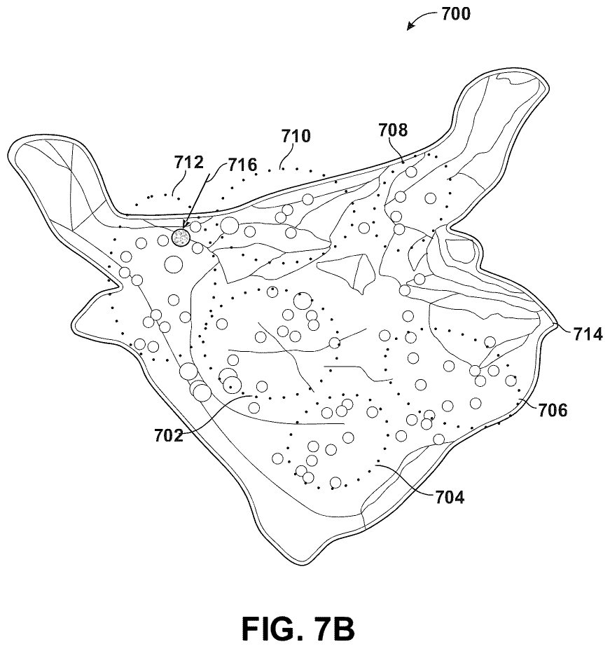

FIG. 7A is an example of a disease baseline map 700 for therapy treatment. In some examples, the map 700 can be provided by a mapping system, such as the mapping system 302, as shown in FIG. 3. Thus, reference can be made to the examples of FIGS. 1-6 in the example of FIG. 7A. In some examples, electrical activity can be rendered on the map 700 in the example of FIG. 7A, however, for clarity and brevity purposes has been omitted. The baseline map 700 includes disease scores having a given color indicating a severity. Disease scores having a greatest severity (e.g., identified using a red color, for example) are identified as 702-712. The disease scores can be computed according to the examples disclosed and rendered on a map to provide the baseline map 700, as shown in FIG. 7A. For example, the disease scores can be computed from electrophysiological signals (e.g., EP signals) recorded on a surface of interest 714, as shown in FIG. 7A. In some examples, the surface of interest is a cardiace surface. In some examples, areas on the surface of interest having a greatest concentration can be identified to identify a number of disease score areas or regions, which can correspond to the 702-712 as shown in FIG. 7B.

FIG. 7B is an example of the disease map 700 updated with one or more feature state changes, which can be associated with a sphere on the surface of interest. Thus, reference can be made to the examples of FIGS. 1-7A in the example of FIG. 7B. The feature state changes (information) can be computed according to the examples disclosed herein. Each sphere can represent an ablation (e.g., of a location or lesion on the surface of interest 714). In the example of FIG. 7B, a feature state change was detected at a corresponding sphere (associated with a location on the surface of interest, or a target site). The detected feature state change is identified with an arrow 716 in the example of FIG. 7B. A severity of the feature state change can be also identified according to a coloring scheme. In some examples, subtle and major feature state changes can be detected according to the examples disclosed herein and assigned a corresponding color to differentiate these changes. Thus, according to the examples disclosed herein, feature state changes can be identified, and represented on a disease score map with a corresponding color to differentiate different types of feature state changes on a map.

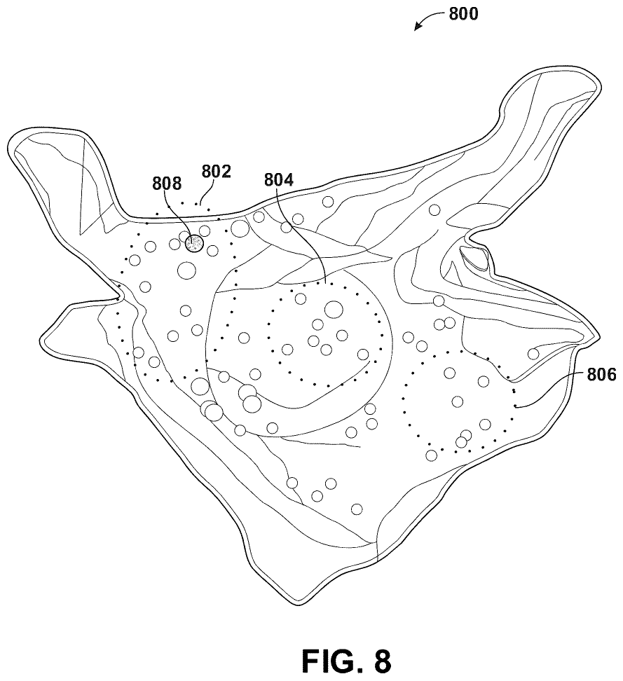

FIG. 8 is an example of the disease map 700 after being updated according examples disclosed herein and is referred to as a disease map 800 e.g. The map 800 can be provided by a mapping system, such as the mapping system 302, as shown in FIG. 3. Thus, reference can be made to the examples of FIGS. 1-7 in the example of FIG. 7. In the example of FIG. 8, the disease map 800 (e.g., points) can be calculated according to the examples disclosed herein (e.g., with a modified disease score equation that emphasizes (e.g., by adjusting one or more feature weights), and thus modified feature coefficients to top disease features seen underlying one or more treatment sites that elicited one or more prior feature state changes. In some examples, electrical activity can be rendered on the map 800, however, for clarity and brevity purposes has been omitted from the map 800.

The map 800 includes disease scores having a given color indicating a severity. Disease scores having a greatest severity (e.g., identified using a red color, for example) are identified as 802-806 (e.g., after ablation based on the map 700, as shown in FIG. 7B). The disease scores can be computed according to the examples disclosed and rendered on a map to provide the map 800. In some examples, areas on the surface of interest having a greatest concentration can be identified to identify a number of disease score areas or regions, which can correspond to the 802-806, as shown in FIG. 8. Feature state changes (information) can be computed according to the examples disclosed herein. Each sphere can represent a prior ablation. In the example of FIG. 8, a feature state change was detected at a corresponding sphere (associated with a location on the surface of interest, or a target site) identified as 808. A severity of the feature state change can be also identified according to a coloring scheme. In some examples, a color of the feature state change can indicate whether a clinician should continue an ablation at the corresponding sphere (e.g., a target site). For example, if the sphere 808 has about a similar color (e.g., visually difficult to differentiate for the clinician) as a sphere of a prior computed map (e.g., the sphere identified with the arrow 716, as shown in FIG. 7B) this can indicate to the clinician to continue ablation at this site on a surface of interest.

### Feature Signal Plots

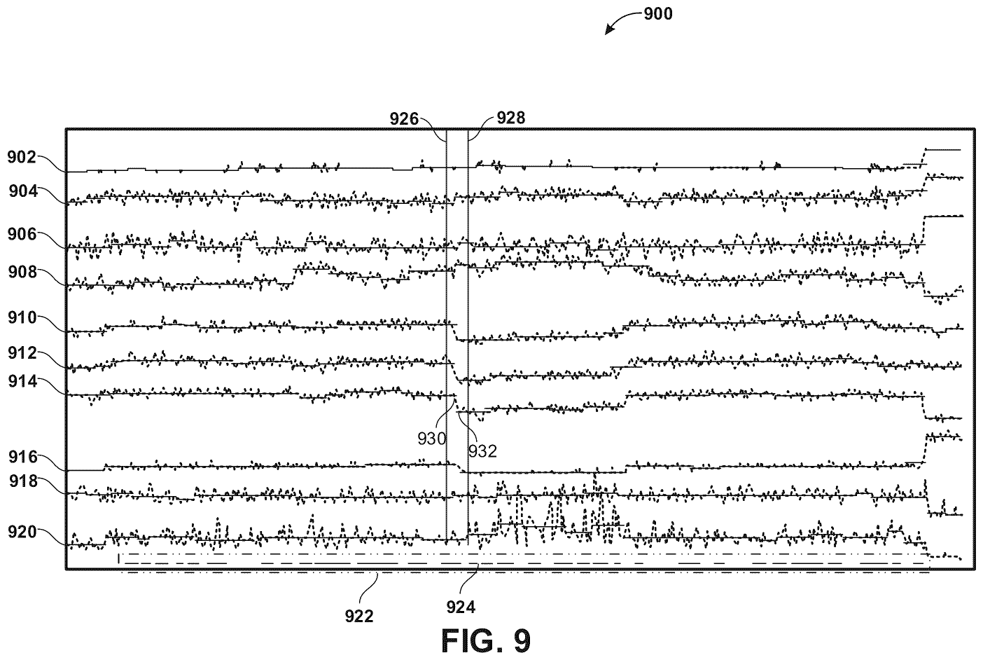

FIG. 9 is an example of a plot 900 of feature signals 902-920 for respective features. In some examples, the feature signals 902-920 can correspond to the feature signals 106, as shown in FIG. 1. Thus, reference can be made to examples of FIGS. 1-8 in the example of FIG. 9. In some examples, the feature signals 902-920 can be provided based on a respective electrophysiological signal (or two or more). The feature detector 100 can provide each of the feature signals 902-920 according to the examples described herein. For each feature signal segment, the feature detector 100 can compute a feature state (identified with a dashed line in the example of FIG. 9). Thus, the FS window 114 (or corresponding FS windows) can be applied to the feature signals 902-920 (e.g., move from left to right) to sample a portion of a feature signal (corresponding to a feature signal segment) and compute the feature state for the sampled portion of the feature signal. For example, during cardiac ablation, a clinician can ablate baseline target sites on a surface of interest within a patient's body that have been identified prior to ablation treatment. At 922 in the example of FIG. 9 horizontal lines are used to illustrate an ablation. A length of each horizontal line at 922 can be indicative of an amount of time that therapy was applied to a corresponding target site identified prior to ablation.

Feature state change data (e.g., the feature state change data 122, as shown in FIG. 1) associated with ablation of a prior target site (identified as 924 in the example of FIG. 9) can be used to identify (or detect) arrhythmogenic activity at another location on the surface of interest within the patient's body. At about a time that ablation was applied to the prior target site is identified at 926 in the example of FIG. 9 and can be referred to herein as a treatment time. At a time after the treatment time the feature state change can change (e.g., in value) from a first feature state value to a second feature state value that is equal to or greater than a feature state change threshold (e.g., state change detection criteria 120, as shown in FIG. 1) at least for some of the feature signals 902-920. In some instances, not all feature signals (e.g., the feature signals 902-920) may change in feature state value so that the feature state change is equal to or greater than the feature state change threshold. This is because some features can be more sensitive than other features. At about a time identified as 928 after the treatment time the feature state for at least some of the feature signals 902-920 can change from the first feature state value to the second feature state value. The time at about which the feature state for at least some of the feature signal 902-920 is equal to or greater than the feature state change threshold can be referred to herein as a feature state change time.

In the example of FIG. 9, feature signals 910-914 are shown as exhibiting a feature state change. According to the examples disclosed herein, in some instances, a threshold number of feature state changes need to be observed before it can be determined that the electrophysiological experienced a feature state change. In some instances, the three feature signals 910-914 can be evaluated to identify a given feature signal having the greatest feature state change according to the examples disclosed herein. The identified given feature and thus for the electrophysiological can be used to identify other electrophysiological signals (associated with locations on the surface of interest) exhibiting a similar or same feature state change according to the examples disclosed herein. The identified other electrophysiological signals or location on the surface of interest can be identified as new potential target sites for treatment.

In some examples, the feature detector 100 computes a difference between the first and second feature state values and compares the difference to threshold to determine whether the difference is equal to or greater than the threshold to detect a feature state change. In other examples, the feature state change detector compares feature state values to the threshold and when a feature state value is equal to or greater than the threshold this can be indicative of a feature state change. As an example, the feature signal 912 has the first feature state value at about a time identified by 930 and a second feature state value at a later time identified by 932 in the example of FIG. 9. The feature detector 100 can detect the feature state change for the feature signals based on the first and second feature state values. As shown in the example of FIG. 9, the feature state for the feature signal 912 changes after the ablation time from the first feature state value to the second feature state value after which at the feature state change time the feature state change can be detected.

### Methods

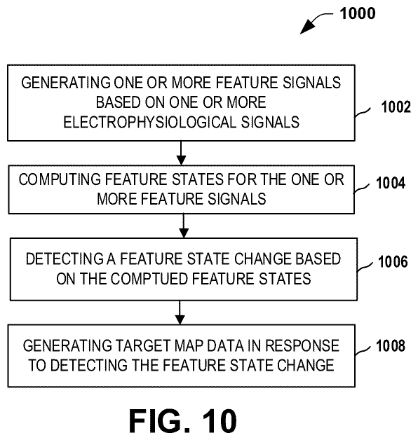

In view of the foregoing structural and functional features described above, example methods will be better appreciated with reference to FIGS. 10-15. While, for purposes of simplicity of explanation, the example methods of FIGS. 10-15 are shown and described as executing serially, it is to be understood and appreciated that the example methods are not limited by the illustrated order, as some actions could in other examples occur in different orders, multiple times and/or concurrently from that shown and disclosed herein.

FIG. 10 illustrates an example of a method 1000 for identifying a target site within a body of a patient. The method 1000 can be implemented by the systems disclosed herein. Therefore, references can be made to the examples of FIGS. 1-9 in the following description of the example of FIG. 10. The method 1000 can begin at 1002 by generating (e.g., by the feature state quantifier 102, as shown in FIG. 1) one or more feature signals (e.g., the feature signals 106, as shown in FIG. 1) based on one or more electrophysiological signals (e.g., the electrophysiological data 104, as shown in FIG. 1) captured from the patient.

At 1004, feature states (e.g., the feature states 116, as shown in FIG. 1) can be computed (e.g., by the feature state quantifier 102) for the one or more feature signals. At 1006, a feature state change can be detected (e.g., by the state change detector 118, as shown in FIG. 1) based on the computed feature states. The feature state change can be indicative of a change in electrical activity on a surface of interest within a patient's body. At 1008, target map data (e.g., the target map data 216, as shown in FIG. 2) identifying a region of interest (e.g., the target site) on the surface of interest can be generated (e.g., by the target generator 206, as shown in FIG. 2) in response to detecting the feature state change. In some examples, at 1008 the method 1000 can include identifying (eg., by the state change detector 118, as shown in FIG. 1, or the target generator 206) common high disease features underlying the treatment(s) that elicited a state change. The disease equation can be biased to highlight signals embodying these high disease feature values at 1008. The target map can be provided at 1008 differentiating (e.g., highlighting) these high disease feature values on a surface of interest (e.g., heart surface).

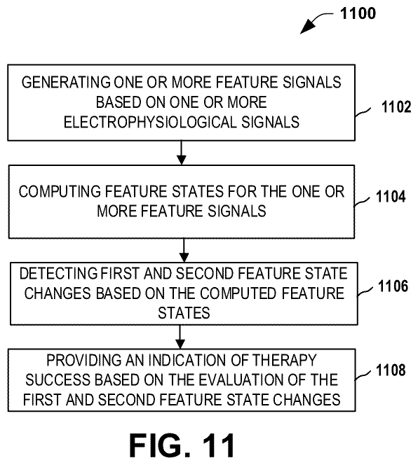

FIG. 11 is an example of a method 1100 for predicting treatment success (e.g., a success of eliminating arrhythmogenic activity). The method 1100 can be implemented by one or more systems disclosed herein. Therefore, references can be made to the examples of FIGS. 1-9 in the following description of the example of FIG. 11. The method 1100 can begin at 1102 by generating (e.g., by the feature state quantifier 102, as shown in FIG. 1) one or more feature signals (e.g., the feature signals 106, as shown in FIG. 1) based on one or more electrophysiological signals (e.g., the electrophysiological data 104, as shown in FIG. 1) captured from the patient. At 1104, feature states (e.g., the feature states 116, as shown in FIG. 1) can be computed (e.g., by the feature state quantifier 102) for the one or more feature signals. At 1106, first and second feature state changes can be detected (e.g., by the state change detector 118, as shown in FIG. 1) based on the computed feature states. At 1108, an indication of therapy success can be provided (e.g., by the state change detector 118) based on an evaluation of the first and second feature state changes (e.g., as they relate to target feature state values and feature state change targets.

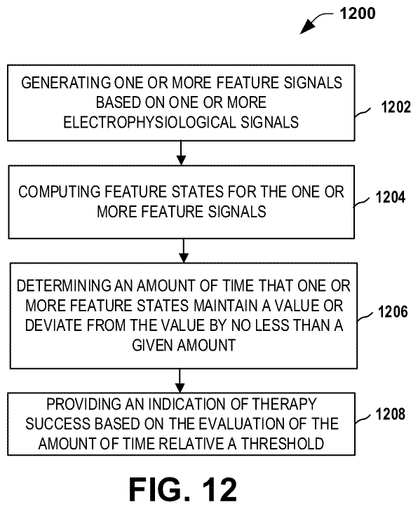

FIG. 12 is an example of another method 1200 for predicting treatment success (e.g., a success of eliminating arrhythmogenic activity). The method 1200 can be implemented by one or more systems disclosed herein. Therefore, references can be made to the examples of FIGS. 1-9 in the following description of the example of FIG. 12. The method 1200 can begin at 1202 by generating (e.g., by the feature state quantifier 102, as shown in FIG. 1) one or more feature signals (e.g., the feature signals 106, as shown in FIG. 1) based on one or more electrophysiological signals (e.g., the electrophysiological data 104, as shown in FIG. 1) captured from the patient. At 1204, one or more feature states (e.g., the feature states 116, as shown in FIG. 1) can be computed (e.g., by the feature state quantifier 102) for the one or more feature signals after application of the therapy to a target site. At 1206, an amount of time that the one or more feature states maintain a value or deviate from the value by no less than a given amount can be determined (e.g., by the state change detector 118, as shown in FIG. 1). At 1208, an indication of treatment success can be provided (e.g., by the state change detector 118) based on an evaluation of the amount of time that the one or more feature states maintain the value or deviates from the value by the given amount relative to a reference value (e.g., the feature state time reference value, as disclosed herein).

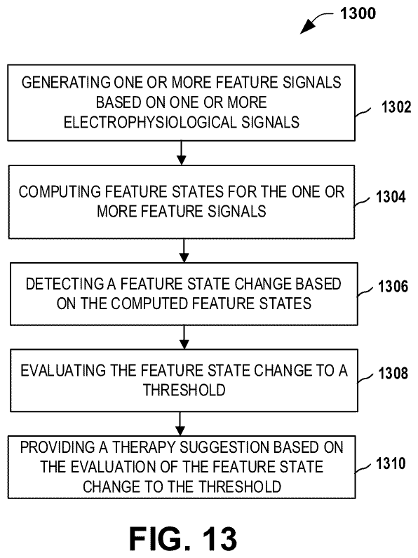

FIG. 13 illustrates an example of a method 1300 for providing a treatment suggestion for a treatment of arrhythmogenic activity. The method 1300 can be implemented by one or more systems disclosed herein. Therefore, references can be made to the examples of FIGS. 1-9 in the following description of the example of FIG. 13. The method 1300 can begin at 1302 by generating (e.g., by the feature state quantifier 102, as shown in FIG. 1) one or more feature signals (e.g., the feature signals 106, as shown in FIG. 1) based on one or more electrophysiological signals (e.g., the electrophysiological data 104, as shown in FIG. 1) captured from the patient. At 1304, feature states (e.g., the feature states 116, as shown in FIG. 1) can be computed (e.g., by the feature state quantifier 102) for the one or more feature signals. At 1306, a feature state change can be detected (e.g., by the state change detector 118, as shown in FIG. 1) based on the computed feature states. At 1308, the feature state change can be evaluated relative to a reference value to determine a relative proximity or distance of the feature state change to the threshold. At 1310, a treatment suggestion can be provided (e.g., by the state change detector 118) based on the relative proximity or distance of the feature state change to the reference value.

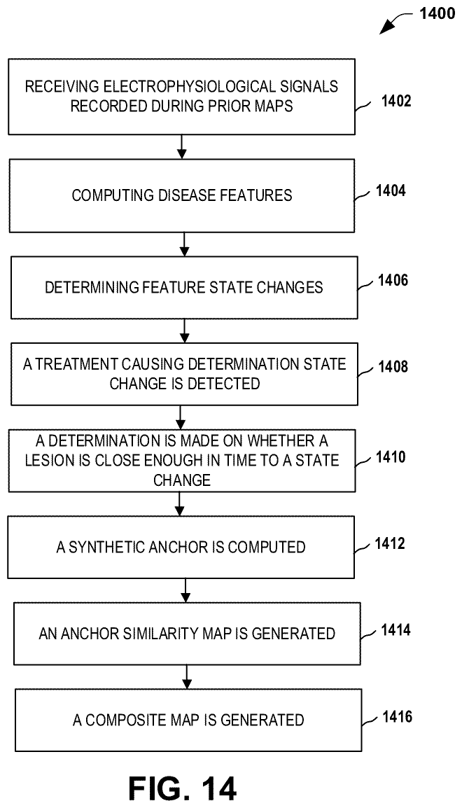

FIG. 14 is an example of a method 1400 for updating treatment targets using information from prior state changes. The method 1400 can be implemented by one or more systems disclosed herein. Therefore, references can be made to the examples of FIGS. 1-9 in the following description of the example of FIG. 14. The method 1400 can begin at 1402 by receiving electrophysiological signals recorded during prior maps. In some examples, map data (e.g., the map data 212, as shown in FIG. 2) is provided at 1402. The map data can include, per map point, electrophysiological signals with start and end times, and corresponding surface of interest locations (e.g., X, Y, Z surface locations). At 1404, disease features (e.g., feature states, such as the feature states 116, as shown in FIG. 1) can be computed across an interval (based on corresponding start and end times) for each electrophysiological signal, at each map point based on the map data.

At 1406, feature state changes are determined based on the disease features. A timestamp associated with each feature state change can also be stored (e.g., in memory, such as disclosed herein). In some examples, feature state change data (e.g., the feature state change data 122, as shown in FIG. 1, or 202, as shown in FIG. 2) is provided at 1406. In some examples, at 1408, treatment data is received. The treatment data can be stored in memory (e.g., as disclosed herein). The treatment data can correspond to the treatment data 134, as shown in FIG. 1, or 208, as shown in FIG. 2. The treatment data can include for each treatment lesion/target site: location information (e.g., X, Y, Z surface locations), and start and end times.

At 1408, a treatment causing determination state change is detected based on the feature state change data and the treatment data. At 1410, a determination can be made whether treatment of a lesion (or target site) is close enough in time to a state change to assume causality. At 1412, a representative synthetic anchor can be computed in response to the detection at 1408. For example, at 1412, feature values for each disease feature from the electrophysiological signals can be computed. The feature values can be viewed along a spectrum from prior mapping. In some examples, at 1410, features can be identified where values are similar enough relative to a value range (e.g., A=2, B=3, range 100), or features can be identified where a subset of values are “similar enough” relative to value range. At 1412, for each entire-group or group-subset, a range of values per feature can be returned that represents the group corresponding to the synthetic anchor (in some examples referred to herein as an anchor signal or anchor point). In some examples, instead of computing synthetic anchors from that case's past state changes, synthetic anchors from past case matches can be used for creating anchor similarity maps, which can be retrieved from a database (e.g., the feature database 124, as shown in FIG. 1).

At 1414, an anchor similarity map can be generated. For example, the map can include points with a color similar to the synthetic anchor. There can be generated one map per anchor group. In some examples, at 1416, a composite map can be generated that aggregates all anchor similarity map data. The composite map can be used to highlight an average similarity value across maps, max, min, etc. In some examples, a custom weighted average of maps can be used. Discussed above are various layers of attributes, e.g., disease features, responses, computing anchor, anchor similarity maps. As more case data denoting which attribute layers/values predict success are collected, coefficients above can be fine tuned.

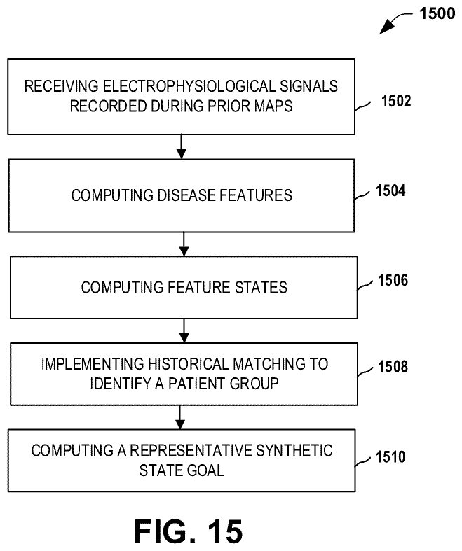

FIG. 15 is an example of a method 1500 for arrhythmia state meta endpoints for optimal success. The method 1500 can be implemented by one or more systems disclosed herein. Therefore, references can be made to the examples of FIGS. 1-9 in the following description of the example of FIG. 15. The method 1500 can begin at 1502 by receiving electrophysiological signals recorded during prior maps. In some examples, map data (e.g., the map data 212, as shown in FIG. 2) is provided at 1502. The map data can include, per map point, (raw) electrophysiological signals with start and end times, and corresponding surface of interest locations (e.g., X, Y, Z surface locations).

At 1504, disease features can be computed across an interval (based on corresponding start and end times) for each electrophysiological signal, at each map point based on the map data. The computed disease features at step 1504 can be stored in a database (e.g, the feature database 124, as shown in FIG. 1). At 1506, feature states can be computed based on the disease features (e.g., feature signals) and can be referred to as baseline feature states. The baseline feature states can be provided as state data. At 1508, a historical matching can be implemented to find patients that have similar regional feature data and state data as a present patient. In some examples, at 1508, a patient group that has a similar enough baseline state and similar enough baseline region feature data can be identified for downstream processing.

At 1510, a representative synthetic state goal can be computed based on the data from step 1508. At 1510, a subgroup of patients in the group of patients from 1508 can be identified with most similar properties. From it, synthetic arrhythmia state goals can be computed and displayed that are most applicable to the current patient, such that if achieved is most likely for long term success. In some examples, at 1512, representative synthetic regional feature data can be computed. At 1512, a subgroup of patients in the group of patients from 1508 can be identified with most similar properties.

FIG. 16 is an example of a plot 1600 of electrophysiological signals that can be processed according to a p-wave localization technique, as disclosed herein. The p-wave localization technique can be implemented by the detector 100. Thus, reference can be made to the example of FIG. 1 in the example of FIG. 16. The plot 1600 includes electrophysiological signals 1602-1614, which are shown as ECG signals. For example, the feature signal generator 108 can receive the electrophysiological signals 1602-1614, which can be provided as part of the electrophysiological data 104, as shown in FIG. 1.

The feature state quantifier 102 can compute for the electrophysiological signal 1602 a number of signal components (identified as a-h in the example of FIG. 16) for a given component or portion of the electrophysiological signal 1602. For example, the feature state quantifier 102 can approximate each signal signal component a-h by ignoring deflections that are less than a deflection amplitude threshold, and where a signal value returns to near an original voltage value over a period of time that is less than a time threshold. While the example of FIG. 16 illustrates the signal components being computed for the electrophysiological signal 1602 it is understood that the feature signal generator 108 computes signal components for each of the remaining electrophysiological signals 1604-1614, as shown in FIG. 16 in a same or similar manner as disclosed herein. Using the signal components, the feature state quantifier 102 can calculate various features on these signals, such as disclosed herein.

For example, across a T-P interval (represented with a dotted line) for each electrophysiological signal 1602-1614 (and thus across each channel), the feature state quantifier 102 can identify components with significant signal deviation (e.g., b, c, f, and h) among all components a-h or from a component(s) exhibiting nearly zero over some time interval. The feature state quantifier 102 can identify a first deviating signal component (e.g., b) and calculate how long (e.g., amount of time) this signal component deviates before returning to a next near-flat component (e.g., d). In the present case, the wavelet can be determined or a function of a start of the first deviating signal component (e.g., b) and an end of a subsequent deviating signal component (e.g., c) (or start of the next near-flat component (e.g., d). Because signal components b and c have enough amplitude (X), max value (Y), and returns to a near-flat component of which has a voltage value similar to signal component a (volt_sim) this can be considered or identified by the feature state quantifier 102 as a wavelet for that channel. The feature state quantifier 102 can identify attributes of the wavelet, for example, the amplitude X, max value Y, and volt_sim. For each of the electrophysiological signals 1602-1614, the feature state quantifier 102 can identify a corresponding wavelet position and its attributes. Furthermore, a cross-channel wavelet instance can be detected based upon the wavelet's attributes for each channel, and the degree of time alignment between attributes.

Accordingly, the feature state quantifier 102 can characterize a p-wave (or wavelet) for each of the electrophysiological signals 1602-1614. In some examples, the feature signal generator 108 can provide each wavelet for each electrophysiological signal. In some examples, the feature signal generator 108 can include a p-wave localizer, such as the p-wave localizer 140, as shown in FIG. 1, that can be configured to implement functionality as disclosed herein for p-wave characterization. The wavelet provided by the p-wave localizer 140 can be processed, for example, by the feature signal generator 108 to generate a feature signal, which can be processed by the feature state calculator 112 to provide a feature state for feature state change detection (e.g., at the state change detector 118, as shown in FIG. 1).

In some examples, the feature state quantifier 102 can determine a fiducial marking (or a centroid) by evaluating a slope according to the equation slope(n)−slope(n−1)>X, where n is a slope of a signal at a given instance of time, and X is the amplitude X of a wavelet. By way of example, the signal components a-b can be identified by the feature state quantifier as a fidicual marking. In some examples, the feature state quantifier 102 can determine a wavelet prominence. In this example, the a-b fiducial marking can be aligned across a number of electrophysiological signals 1602-1614, in the example of FIG. 16, the a-b fiducial marking is aligned 6 out of 7 of the electrophysiological signals 1602-1614. In each, slope(a) is near zero followed by a significantly [non-zero slope (b) and a long time to return to volt_sim(A) and max_volt_diff from A during the wavelet>X]. For example, the numerator can be calculated as abs((slop(n)−slope(n−1)), time (return_to_volt_sim(n−1))−time(onset-fiducial)), and/or abs(max_volt_wavelet−volt_n−1). The denominator can be calculated as slope(n−1). Fiducial time aligmenet can be implemented by the feature state quantifier 102. For example, using the parameters above, the feature state quantifier 102 can calculate how similar in time each type of fiducial appears across signals. In this example, the a-b fiducial marking is aligned across six channels. The stdev or interquartile ratio can be about zero, and if there was no fiducial aligment, then the value would be great. The time aligment feature includes onset fiducal aligment, nearest fidicuial aligment, and peak/trough fiducial aligment, as well as others. In some examples, the feature state quantifier evaluates the attribute aligment of onset fidicular. In this example, the a-b fiducial marking attributes are compared to e-f fiducial marking attributes. The more similar the marking attributes are beat-to-beat means “progress” by way of increased organization or electrical synchrony across the heart. Furthermore, p-wave (or wavelet) instances with a high degree of attribute alignment can be more accurately localized via the rhythm localizer.

### Further Computer-Implemented Embodiments and Example Implementations

Various embodiments (including the feature detector 100 and its components, the system 200, as shown in FIG. 2, the system 300, as shown in FIG. 3, the analysis system 424, as shown in FIG. 4 can be implemented on one or more computing devices. The computing devices may be at the same or different locations. A computing device can be any type of device having one or more processors and memory. For example, a computing device can be a workstation, mobile device (e.g., a mobile phone, personal digital assistant, tablet or laptop), computer, server, computer cluster, server farm, game console, set-top box, kiosk, embedded system, or other device having at least one processor and computer-readable memory. In addition to at least one processor and memory, such a computing device may include software, firmware, hardware, or a combination thereof. Software may include one or more applications and an operating system. Hardware can include, but is not limited to, a processor, memory and user interface display or other input/output device.

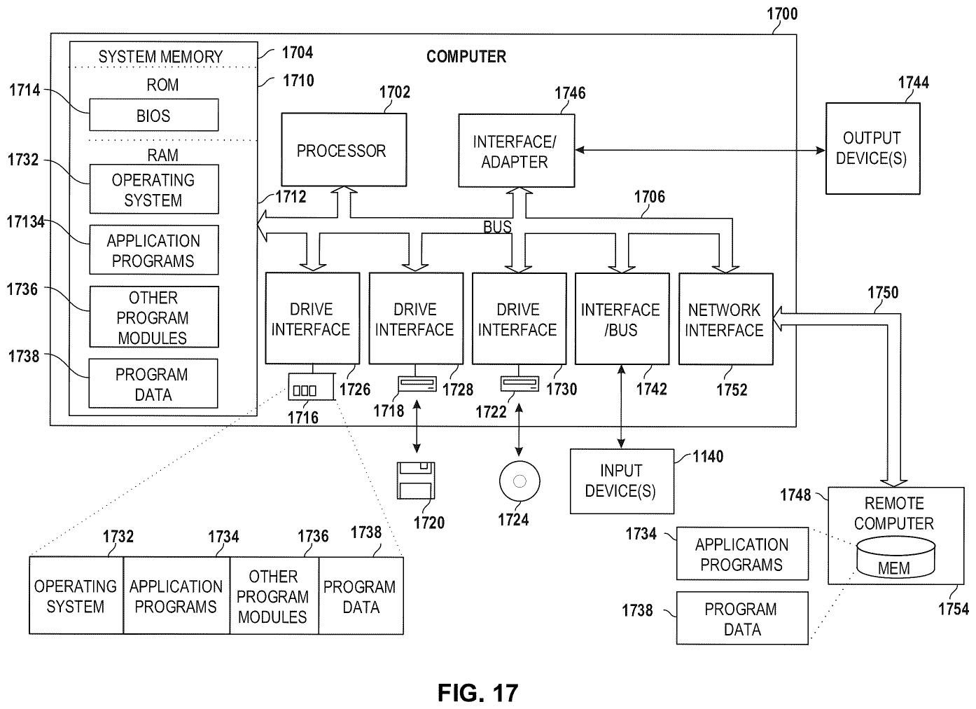

In view of the foregoing structural and functional description, those skilled in the art will appreciate that portions of the embodiments can be embodied as a method, data processing system, or computer program product. Accordingly, these portions of the present embodiments can take the form of an entirely hardware embodiment, an entirely software embodiment, or an embodiment combining software and hardware, such as shown and described with respect to the computer system of FIG. 17. Furthermore, portions of the embodiments can be a computer program product on a computer-usable storage medium having computer readable program code on the medium. Any non-transitory, tangible storage media possessing structure can be utilized including, but not limited to, static and dynamic storage devices, hard disks, optical storage devices, and magnetic storage devices, but excludes any medium that is not eligible for patent protection under 35 U.S.C. § 101 (such as a propagating electrical or electromagnetic signal per se).

As an example and not by way of limitation, a computer-readable storage media can include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium can be volatile, nonvolatile, or a combination of volatile and non-volatile, where appropriate.

Certain embodiments have also been disclosed herein with reference to block illustrations of methods, systems, and computer program products. It will be understood that blocks of the illustrations, and combinations of blocks in the illustrations, can be implemented by computer-executable instructions. These computer-executable instructions can be provided to one or more processor of a general-purpose computer, special purpose computer, or other programmable data processing apparatus (or a combination of devices and circuits) to produce a machine, such that the instructions, which execute via the processor, implement the functions specified in the block or blocks.

These computer-executable instructions can also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions can also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

In this regard, FIG. 17 illustrates one example of a computer system 1700 that can be employed to execute one or more embodiments of the present disclosure. Computer system 1700 can be implemented on one or more general purpose networked computer systems, embedded computer systems, routers, switches, server devices, client devices, various intermediate devices/nodes or standalone computer systems. Additionally, computer system 1700 can be implemented on various mobile clients such as, for example, a personal digital assistant (PDA), laptop computer, pager, and the like, provided it includes sufficient processing capabilities.

Computer system 1700 includes processing unit 1702, system memory 1704, and system bus 1706 that couples various system components, including the system memory 1704, to processing unit 1702. Dual microprocessors and other multi-processor architectures also can be used as processing unit 1702. System bus 1706 may be any of several types of bus structure including a memory bus or memory controller, a peripheral bus, and a local bus using any of a variety of bus architectures. System memory 1704 includes read only memory (ROM) 1710 and random access memory (RAM) 1712. A basic input/output system (BIOS) 1714 can reside in ROM 1710 containing the basic routines that help to transfer information among elements within computer system 1700.

Computer system 1700 can include a hard disk drive 1716, magnetic disk drive 1718, e.g., to read from or write to removable disk 1720, and an optical disk drive 1722, e.g., for reading CD-ROM disk 1724 or to read from or write to other optical media. Hard disk drive 1716, magnetic disk drive 1718, and optical disk drive 1722 are connected to system bus 1706 by a hard disk drive interface 1726, a magnetic disk drive interface 1728, and an optical drive interface 1730, respectively. The drives and associated computer-readable media provide nonvolatile storage of data, data structures, and computer-executable instructions for computer system 1700. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and disclosed herein.

A number of program modules may be stored in drives and RAM 1710, including operating system 1732, one or more application programs 1734, other program modules 1736, and program data 1738. In some examples, the application programs 1734 can include the detector 100, as shown in FIG. 1, the system 200, as shown in FIG. 2, the system 300, as shown in FIG. 3, and/or the analysis system 424, as shown in FIG. 4. The application programs 1734 and program data 1738 can include functions and methods programmed for, for example, target site identification, treatment suggestion, and treatment success.

A user may enter commands and information into computer system 1700 through one or more input devices 1740, such as a pointing device (e.g., a mouse, touch screen), keyboard, microphone, joystick, game pad, scanner, and the like. These and other input devices are often connected to processing unit 1702 through a corresponding port interface 1742 that is coupled to the system bus, but may be connected by other interfaces, such as a parallel port, serial port, or universal serial bus (USB). One or more output devices 1744 (e.g., display, a monitor, printer, projector, or other type of displaying device) is also connected to system bus 1706 via interface 1746, such as a video adapter.

Computer system 1700 may operate in a networked environment using logical connections to one or more remote computers, such as remote computer 1748. Remote computer 1748 may be a workstation, computer system, router, peer device, or other common network node, and typically includes many or all the elements described relative to computer system 1700. The logical connections, schematically indicated at 1750, can include a local area network (LAN) and a wide area network (WAN). When used in a LAN networking environment, computer system 1700 can be connected to the local network through a network interface or adapter 1752. When used in a WAN networking environment, computer system 1700 can include a modem, or can be connected to a communications server on the LAN. The modem, which may be internal or external, can be connected to system bus 1706 via an appropriate port interface. In a networked environment, application programs 1634 or program data 1738 depicted relative to computer system 1700, or portions thereof, may be stored in a remote memory storage device 1754.

FIG. 18 is an example of a plot 1800 of mapping points. A line 1808 in the plot 1800 can represent a composite feature state value computed for each time interval based on electrophysiological signal(s) recorded (e.g., at a location) across X time, which is 30 minutes in this example (other timing intervals are contemplated). Each state value and thus composite feature state value can be computed by the detector 100, as shown in FIG. 1. Thus, reference can be made to one or more examples of FIGS. 1-17 in the example of FIG. 18. Each line segment A-C of the line 1808 represents a unique composite feature state value, where uniqueness occurs when a newly presenting state deviates beyond a certain percentage or absolute value tolerance compared to another state.

In the example of FIG. 18, an arrhythmia has two distinct instances of state A, two of state B, and one of state C that occurred during the time interval. During this time interval, mapping data (corresponding to electrophysiological signals and their location) can be recorded (or captured) from a heart's surface (or surface of interest), either by a catheter at various heart points, single beat contact or non-contact mapping techniques from the outside or inside, or from other alike sources. These mapping points are pictorially denoted with dot points above an x-axis for each time instance that a mapping point was generated in this example (these points may be continuous in the single beat mapping scenario). Each dot point can have a unique color and each dot color can indicate which state value each map point was recorded during. Thus, the plot 1800 can include a number of dot points 1802-1806 that can be associated with a corresponding state (e.g., state A, state B, or state C).

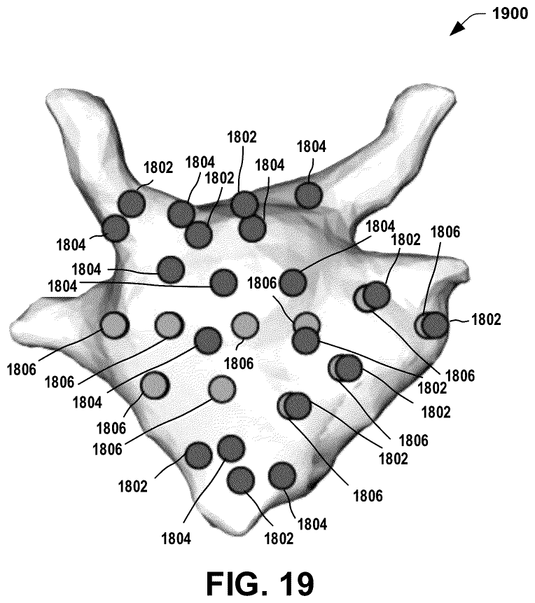

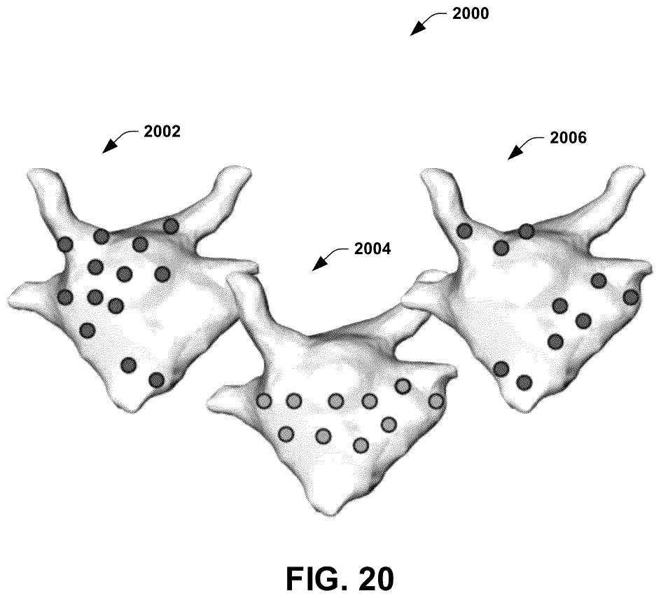

FIG. 19 is an example map 1900 with mapping points based on a mapping point plot, such as the plot 1800, as shown in FIG. 18. Thus, reference can be made to one or more examples of FIGS. 1-18 in the example of FIG. 19. FIG. 19 illustrates a map point distribution on a surface of interest (e.g., the heart;s surface) when all points are superimposed onto a single map. In this example, disease feature values (computed according to the examples disclosed herein) can be an average, median, maximum, etc value from all map points at that point or region, which can be provided as part of target map data (e.g., the target map data 216, as shown in FIG. 2). Additionally, in another example, disease feature values can first calculate the average, median, maximum, etc at that point or region for each observed State (A-C in this example), and can subsequently be used for later calculations, for example, as disclosed herein (e.g., with respect to FIG. 20).

FIG. 20 are example maps 2002-2006 with mapping points per feature state based on a mapping point plot, such as the plot 1800, as shown in FIG. 18. Thus, reference can be made to one or more examples of FIGS. 1-19 in the example of FIG. 19. For example, the map 2002 is shown with the mapping points 1802, the map 2004 is shown with the mapping points 1804, and the map 2006 is shown with the mapping points 1806.

Accordingly, the present technology can leverage simultaneous composite feature state values per map point to generate map and disease score data. For example, during the mapping stage the calculation and display of mapping points per simultaneous composite feature state can indicate to the clinician which heart regions are missing mapping points per each composite state, and/or for all composite states that have been seen during mapping. Furthermore, after the clinician (or user) completes the mapping step (or electrophysiological recording step), the detector 100 is able to cause disease scores to be displayed to the user on an output device and target map data associated with a single, or subset of, all states. In one example, the detector 100 can cause composite disease score data to be displayed on the output device that is a time weighted sum of all disease scores observed at a location (e.g., X, Y, Z location) or region. For example, where state A=20 minutes (min), State B=5 min, and State C=5 min, a k location score or region score could be ⅔*map-A-disease-score+⅙*map-B-disease score+⅙*map-C-disease-score (which would not be otherwise possible to discern the time weights without the known simultaneous composite feature states). Alternatively, in another embodiment, the detector 100 can cause disease score values, or time weighted summations mentioned above, calculated from one or a subset of composite feature states to be rendered on the output device (for the user). For example, the clinical can view the target map data from only the most severe composite state, least severe composite state instance, etc. Furthermore, in another example, a relative (or normalized) disease score can be calculated which can be defined as the disease score associated with mapping point(s) divided by, or normalized to, the composite feature state value simultaneous to that map point.

FIG. 21 is an example of a method 2100 for predicting procedure success conditions. The method 2100 can be implemented by the detector 100, as shown in FIG. 1. Thus, reference can be made to one or more examples of FIGS. 1-20 in the example of FIG. 21. In some examples, the method 2100 can be implemented by a state dynamic calculator, which can be implemented in some instances as part of the feature state calculator 112 or the state change detector 118, in other examples, as a separate module, as part of one or more systems, as disclosed herein.

The method 2100 can begin at 2102, by creating a feature state matrix 2104, as shown in FIG. 21. For example, the feature state values (e.g., individual feature states and/or composite feature states), their underlying feature statistics such as median, mean, stdev, interquartile ratio, variance, and other related statics, and the start and end times for each state can be stored in the feature state matrix 2104. In some examples, these can be thought of as a cumulation of all states that have occurred, across all features, throughout the procedure data recorded. The feature state values can correspond to the feature states 116, as shown in FIG. 1, in some examples.

At 2106, from this state matrix, state dynamics can be calculated that quantify attributes of both the state data across time, the state relationships across features, and the combination thereof. For example, a state dynamics feature could include, but is not limited to, an amount of consecutive times a rhythm is at a unique state compared to all prior state values, the number of unique states presenting*consecutive time at each, for each unique state in the state-value-n−state-value-nearest and/or the cumulation of such values.

In some examples, state dynamic features can include features such as, but not limited to, variation (such as standard deviation, interquartile range or dispersion, or the alike) of state values for a specific time interval, normalized to values from prior time interval data such as pre-ablation time intervals. Thus, the state dynamics features can correspond to or be representative of attributes derived from the prior feature state characteristics, and thereby represent various patterns of the states.

At 2108, the state dynamics are analyzed that predicts, based on the multitude of calculated state dynamics for a given patient, a percent likelihood for a success condition if treatment stopped at this present moment. Success conditions, for example, can include, but are not limited to, X month arrhythmia freedom or atrial fibrillation following the procedure, X % AF burden or arrhythmia burden over Y-Z time period following the procedure, etc.

In some examples, the state dynamics are computed or are a function of the state dynamics (computed at step 2106), map data 2110, localizer data 2112, and treatment data 2114 all of which that can be considered at step 2108 in predicting a likelihood of a success condition. The map data 2110, the localizer data 2112, and the treatment data 2114 can correspond to the map data 130, the localizer data 142, and the treatment data 134, as shown in FIG. 1.

Each state dynamics feature can have a coefficient (or weighting) that represents a type of importance or relationship each has, or a combination of dynamics have, on a success/goal predictor. These coefficients can be trained on historical case datasets using a ML model that, via techniques such as reinforcement learning, learn the contributions, and combination of contributions, that state dynamics have in predicting a specific success condition. As an example, it can be that the ML model learns that patients exhibiting unique state values (relative to prior) for a longer period of time predict an increased likelihood of X month arrhythmia freedom following the procedure.

Additionally, both target map data (including its underlying map data and rhythm localizer data on the heart surface) and the treatment data can be inputs to the success/goal predictor. The relationships between these variables and the ultimate success/goal prediction are learned from the historical datasets. As an example, it can be true that treating X % of target map locations that elicit X state changes presenting a new unique state, with a composite state value Z at present, is associated with a 90% likelihood that success, in this example defined as X month arrhythmia freedom following the procedure, is achieved if stopping the procedure at this present time.

Additionally, the success/goal predictor can define goals of treatment data or state dynamics whose achievement will each result in X % likelihood of the success condition. These goals are derived similar to the above prediction algorithm method(s), but instead of being only applied on data in the present moment, an algorithm (e.g., implemented as part of the detector 100, as shown in FIG. 1, or as part of one or more systems as disclosed herein) searches for the next closest goal, aligned with historical datasets, that predicts X % success, according to a ML model trained from historical datasets. For example, the algorithm may return an 80% likelihood if ablation can elicit a composite state value from 5 (current value) to a goal of 7, or a 90% likelihood if both a) achieve a value of 8, b) change a state value X to any value other than its current.

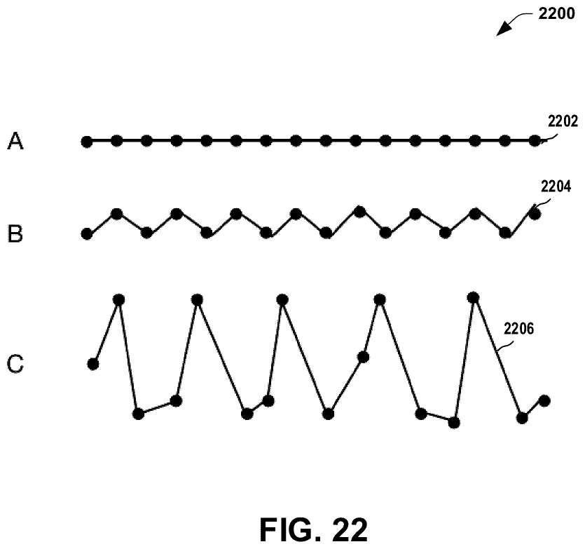

FIG. 22 is an example of different types of feature signals 2202-2206 that underlies a feature state. Dots in FIG. 22 can represent a feature value calculated for each sampling window 110 according to the examples disclosed herein, for example, by the detector 100, as shown in FIG. 1. Thus, reference can be made to one or more examples of FIGS. 1-21 in the example of FIG. 22. For example, row A shows that the feature signal has a near-similar value for each successive sampling window 110, which would thereby indicate to a clinician (e.g., on an output device, as disclosed herein) that the physician needs to record mapping data for a given (e.g., 1) sampling window duration (e.g., 4 seconds) when this feature signal and feature state is present. In another example, row B shows that the feature signal is periodic, or begins to repeat itself, every given (e.g., 2) sampling windows 110, thereby indicating to a clinician (e.g., on the output device) that the clinician needs to record mapping data (e.g., electrophysiological signals) for the given sampling window(s) (e.g., 8 sec). And in row C, using the same logic for a given sampling window(s) (e.g., 12 sec). This calculation of a number of samples until the feature state is periodic is displayed to the clinician (e.g., on the output device) in response to the detector 100, which thereby ensures that mapping data is collected during all cycles of the rhythm's feature state. For example, the detector 100 can evaluate values of features signals to identify one or more consistent feature signals (e.g., as shown in row A), one or more periodic feature signals (e.g., as shown in row B), and nonperiodic/inconsistent one or more feature signals (e.g., as shown in row C). The detector 100 can output signal alert data indicating that the feature state state is periodic or unchanging to alert the clinician. As such, the detector 100 can calculate or determine an amount of time elapsed until feature signal data repeats itself. This time elapsed number can be provided in some instances as the signal alert data and rendered on the output device for the clinician during a mapping phase. For example, the clinician can place a catheter at a location on a surface of interest (e.g., the heart's surface) and thereby knows how long to record data before moving to the next point. The detector 100 can provide this visually independently, or can provide this information to a 3D mapping system (in some instances a mapping system, as disclosed herein) in a procedure room that can integrate this data with their 3D mapping visualization.

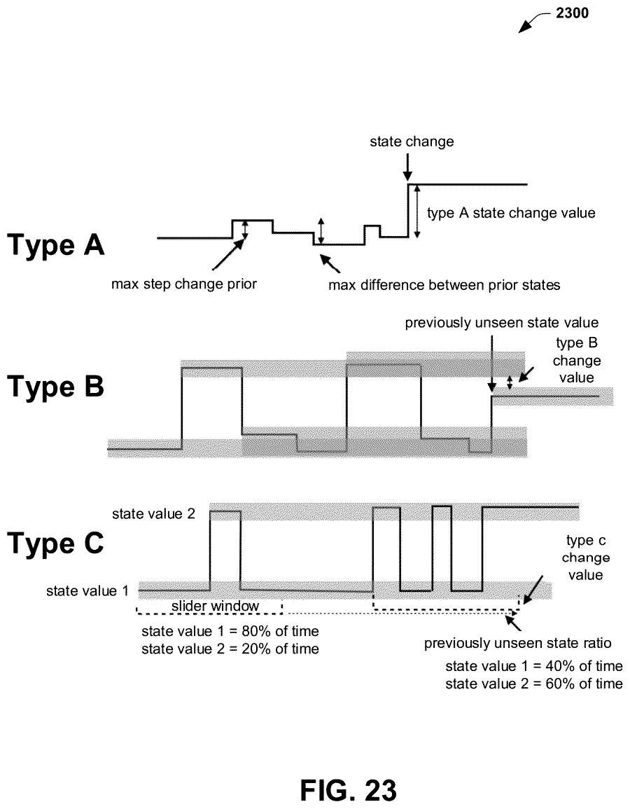

FIG. 23 is an example of different state changes that can be detected or identified by a detector, such as the detector 100, as shown in FIG. 1. Thus, reference can be made to one or more examples of FIGS. 1-22 in the example of FIG. 23. In the example of FIG. 23, three (3) different types of state changes that can be detected (or identified) are shown, and referred to respectively, as “Type A,” “Type B,” and “Type C.” The detector 100, as disclosed herein, can detect or identify different state changes for feature states and composite feature states.

In some examples, Type A is a state change where there is a step change from state n−1 to state n whose amplitude is greater than a threshold. This threshold can be a multiple of the max state step change detected prior (e.g., during baseline or anytime prior). Alternatively, this threshold can be a multiple of the max difference between all states detected prior (e.g., during baseline interval or anytime prior). In this instance, where a Type A state change is detected (e.g., by the detector 100), the assigned value to this detection can be, for example, either the absolute state change value, the state change value normalized to max step change prior, the state change value normalized to the max difference between prior states, or some combination thereof.

In some examples, Type B is a state change type where a present state value has not been previously seen. In this example, baseline data or pre treatment data can be recorded. These states can be the initial “previously seen” states. Once treatment starts, the detector 100 can search (in real-time) for emergence of new states that are previously unseen, as defined by: value of present state—value of next closest state value seen previously >X threshold. FIG. 23 indicates this detected Type B phenomenon. The value associated with this state change is a difference between existing state and the most similar prior state, and can be normalized to a variation or tolerance that bounds each of these states.

In some examples, Type C is a state change type where, over some slider time window (time interval), there is a previously unseen state ratio where the ratio values represent the time occurrence of various states changes beyond a threshold. The threshold is derived from prior state ratio values across all prior slider window intervals and defines a lower boundary of a given state ratio value and a upper boundary of a different state ratio value. For example, the lower boundary can be 70%/30% (e.g., percentage of state value 1 to a percentage of state value 2), and on the upper bound can be 90%/10% (e.g., percentage of state value 1 to a percentage of state value 2). In this example, the prior state value 1 occurred on average 80% of the time and state value 2 occurred on average 20% of the time. As the slider window moves forward in time and encapsulates new state data, it encapsulates state data where the ratio is different (e.g., beyond some threshold, either less than 70%/30% or more than 90%/10% for state1/state2 ratio) than the previously seen ratio. In the second slider window shown in FIG. 23, the state value 1 exists 40% of the time, and state value 2 exists 60% of the time, which lies outside of the upper (90%/10%) and lower (70%/30%) boundaries. This indicates that a state change Type C has occurred, and indicates to a clinician (and all outputs of the state change detector) that an immediate change occurred sometime during this time window to which the slider window spans. The value associated with this state change can be, for example, the state with maximum time occurrence change between present slider window and previous slider window.

The present disclosure is also directed to the following exemplary embodiments:

Embodiment 1: One or more non-transitory computer-readable media having data and machine readable instructions executable by a processor, the data comprising electrophysiological data captured from a patient, the machine readable instructions comprising: a feature state quantifier to compute feature states based on feature signals, the feature signals being generated based on the electrophysiological data; and a state change detector to detect a feature state change indicative of a change in electrical activity on a surface of interest within a patient's body.

Embodiment 2: The one or more non-transitory computer-readable media of embodiment 1, wherein the method further comprises a target generator to output target map data based on the detected feature state change, the target map data identifying a location on the surface of interest within the patient's body.

Embodiment 3: The one or more non-transitory computer-readable media of any embodiments of 1-2, wherein the state change detector is further to: provide data characterizing a set of feature states computed over a period of time; and predict a likelihood of procedure success based on the data.

Embodiment 4: The one or more non-transitory computer-readable media of any embodiments of 1-3, wherein the feature state change is a first feature state change, and the location is a first location, and the first feature state change being detected after therapy at a second location on the surface of interest within the patient's body during the treatment, and wherein the state change detector is to detect a second feature state change before the therapy and output treatment success data indicating a treatment success based on the first and second feature state changes.

Embodiment 5: The one or more non-transitory computer-readable media of embodiment 4, wherein the state change detector is to compute a difference between the first and second feature state changes, and the difference being indicative of the treatment success.

Embodiment 6: The one or more non-transitory computer-readable media of any embodiments of 1-5, wherein the state change detector: determines an amount of time that a feature state computed based on a respective feature signal of the feature signals maintains a value or deviates from the value by a given amount; and evaluates the determined amount of time relative to a feature state time reference to determine a treatment success of a treatment to the patient.

Embodiment 7: The one or more non-transitory computer-readable media of embodiment 6, wherein the state change detector causes the treatment success to be rendered on a display to modify the treatment being applied to the patient.

Embodiment 8: The one or more non-transitory computer-readable media of embodiment 7, wherein the treatment success is determined based on a proximity of the determined amount of time to the feature state time reference.

Embodiment 9: The one or more non-transitory computer-readable media of any embodiments of 1-8, wherein the state change detector evaluates the feature state change relative to a threshold and provides a treatment suggestion based on the evaluation, the treatment suggestion indicating whether a clinician is to continue applying therapy to one or more target sites during a treatment.

Embodiment 10: The one or more non-transitory computer-readable media of embodiment 9, wherein the state change detector causes the treatment suggestion to be rendered on a display.

Embodiment 11: The one or more non-transitory computer-readable media of any embodiments 1-10, wherein the feature state quantifier computes the feature states based on a feature signal segment from one of the feature signals.

Embodiment 12: The one or more non-transitory computer-readable media of embodiment 11, wherein the feature state quantifier is to compute a number of feature values for each feature based on respective portions of electrophysiological signals of the electrophysiological data.

Embodiment 13: The one or more non-transitory computer-readable media of any of embodiments 1-12, wherein the state change detector is to: evaluate a state ratio representing a time occurrence of states over a period of time relative to a threshold; and detect the feature state change in response to the state ratio being equal to or greater than the threshold.

Embodiment 14: The one or more non-transitory computer-readable media of any embodiments 1-12, wherein the state change detector is to: detect feature states corresponding to first feature states; detect a given feature state; and evaluate a respective value of one of the first feature states and the given feature state relative to a threshold to detect the feature state change, wherein a value of the feature state change is a difference between the given feature state and one of the first feature states that is nearest in value to the given feature state.

Embodiment 15: A system comprising memory configured to store machine readable instructions and data comprising electrophysiological data representing electrophysiological signals captured from a patient during a treatment; at least one processor configured to access the memory and configured to execute the machine readable instructions, the machine readable instructions comprising: a feature state quantifier comprising: a feature signal generator to compute a number of feature values for features based on respective electrophysiological signals, and combine the feature values for each feature to generate feature signals; a feature state calculator to compute feature states based on a feature signal segment from a respective feature signal of the feature signals; a state change detector to detect a feature state change indicative of a change in electrical activity on a surface of interest within a patient's body; and a target generator to output target map data based on the detected feature state change, the target map data identifying a location on the surface of interest within the patient's body.

Embodiment 16: The system of embodiment 15, wherein the target generator is to modify a graphical map for the patient to include a graphical element identifying the location on the surface of interest within the patient's body.

Embodiment 17: The system of any of embodiments 15-16, wherein the the machine readable instructions comprise a state dynamic calculator to create a feature state matrix based on at least the feature states; compute state dynamics based on the feature state matrix; and predict a likelihood of procedure success based on the computed state dynamics.

Embodiment 18: A computer-implemented method comprising: receiving, by a processor, electrophysiological data captured from a patient during a therapy treatment of a target site identified prior to a treatment, the target site corresponding to a potential ablation site on a surface of interest within a patient's body; generating, by the processor, feature signals based on the electrophysiological data captured from the patient; computing, by the processor, feature states based on the feature signals; detecting, by the processor, a respective feature state change based on an evaluation of the feature states relative to state change detection criteria; and outputting, by the processor, target map data identifying a region of interest on a surface of interest.

Embodiment 19: The computer-implemented method of embodiment 18, wherein the region of interest has signal feature values that are similar to signal feature values at a prior treated location which elicited a detected arrhythmia state change.

Embodiment 20: The computer-implemented method of any of the embodiments 18-19, further comprising: computing, by the processor, a number of feature values for each feature based on respective portions of electrophysiological signals of the electrophysiological data; combining, by the processor, the feature values for each feature to provide the feature signals; and computing, by the processor, the feature states based on a feature signal segment from one of the feature signals.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

As used herein, the term “or” is intended to be inclusive, rather than exclusive. Unless specified otherwise, “X employs A or B” is intended to mean any of the natural incisive permutations. That is, if X employs A; X employs B; or X employs both A and B, the “X employs A or B” is satisfied. As used herein, the terms “example” and/or “exemplary” are utilized to delineate one or more features as an example, instance, or illustration. The subject matter disclosed herein is not limited by such examples. Additionally, any aspects, features, and/or designs disclosed herein as an “example” or as “exemplary” are not necessarily intended to be construed as preferred or advantageous. Likewise, any aspects, features, and/or designs disclosed herein as an “example” or as “exemplary” is not meant to preclude equivalent embodiments (e.g., features, structures, and/or methodologies) known to one of ordinary skill in the art.

Understanding that is not possible to describe each and every conceivable combination of the various features (e.g., components, products, and/or methods) disclosed herein, one of ordinary skill in the art can recognize that many further combinations and permutations of the various embodiments disclosed herein are possible and envisaged. Furthermore, as used herein, the terms “includes,” “has,” “possesses,” and/or the like are intended to be inclusive in a manner similar to the term “comprising” as interpreted when employed as a transitional word in a claim.

What have been described above are examples. It is, of course, not possible to describe every conceivable combination of components or methods, but one of ordinary skill in the art will recognize that many further combinations and permutations are possible. Accordingly, the present disclosure is intended to embrace all such alterations, modifications, and variations that fall within the scope of this application, including the appended claims. Where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, the term “including” means including but not limited to. The term “based on” means “based at least in part on.”

What is claimed is:

1. One or more non-transitory computer-readable media having data and machine readable instructions executable by a processor, the data comprising electrophysiological data captured from a patient, the machine readable instructions comprising:
a feature state quantifier to compute feature states based on feature signals, the feature signals being generated based on the electrophysiological data; and
a state change detector to detect a feature state change indicative of a change in electrical activity on a surface of interest within a patient's body.

2. The one or more non-transitory computer-readable media of claim 1, further comprising a target generator to output target map data based on the detected feature state change, the target map data identifying a location on the surface of interest within the patient's body.

3. The one or more non-transitory computer-readable media of claim 2, wherein the state change detector is further to:
provide data characterizing a set of feature states computed over a period of time; and
predict a likelihood of procedure success based on the data.

4. The one or more non-transitory computer-readable media of claim 2, wherein the feature state change is a first feature state change, and the location is a first location on the surface of interest, and the first feature state change being detected after therapy at a second location on the surface of interest within the patient's body during the treatment, and wherein the state change detector is to output treatment success data predicting a treatment success based on the first and second feature state changes.

5. The one or more non-transitory computer-readable media of claim 4, wherein the state change detector is to compute a difference between the first and second feature states, and the difference being indicative of the treatment success.

6. The one or more non-transitory computer-readable media of claim 1, wherein the state change detector:
determines an amount of time that a feature state computed based on a respective feature signal of the feature signals maintains a value or deviates from the value by a given amount; and
evaluates the determined amount of time relative to a feature state time reference to determine a treatment success of a treatment to the patient.

7. The one or more non-transitory computer-readable media of claim 6, wherein the state change detector causes the treatment success to be rendered on a display to modify the treatment being applied to the patient.

8. The one or more non-transitory computer-readable media of claim 7, wherein the treatment success is determined based on a proximity of the determined amount of time to the feature state time reference.

9. The one or more non-transitory computer-readable media of claim 1, wherein the state change detector evaluates the feature state change relative to a threshold and provides a treatment suggestion based on the evaluation, the treatment suggestion indicating whether a clinician is to continue applying therapy to one or more target sites during a treatment.

10. The one or more non-transitory computer-readable media of claim 9, wherein the state change detector causes the treatment suggestion to be rendered on a display.

11. The one or more non-transitory computer-readable media of claim 1, wherein the feature state quantifier computes the feature states based on a feature signal segment from one of the feature signals.

12. The one or more non-transitory computer-readable media of claim 11, wherein the feature state quantifier is to compute a number of feature values for each feature based on respective portions of electrophysiological signals of the electrophysiological data.

13. The one or more non-transitory computer-readable media of claim 1, wherein the state change detector is to:
evaluate a state ratio representing a time occurrence of states over a period of time relative to a threshold; and
detect the feature state change in response to the state ratio being equal to or greater than the threshold.

14. The one or more non-transitory computer-readable media of claim 1, wherein the state change detector is to:
detect feature states corresponding to first feature states;
detect a given feature state; and
evaluate a respective value of one of the first feature states and the given feature state relative to a threshold to detect the feature state change, wherein a value of the feature state change is a difference between the given feature state and one of the first feature states that is nearest in value to the given feature state.

15. A system comprising:
memory configured to store machine readable instructions and data comprising electrophysiological data representing electrophysiological signals captured from a patient during a treatment;
at least one processor configured to access the memory and configured to execute the machine readable instructions, the machine readable instructions comprising:
a feature state quantifier comprising:
a feature signal generator to compute a number of feature values for features based on respective electrophysiological signals, and combine the feature values for each feature to generate feature signals;
a feature state calculator to compute feature states based on a feature signal segment from a respective feature signal of the feature signals;
a state change detector to detect a feature state change indicative of a change in electrical activity on a surface of interest within a patient's body; and
a target generator to output target map data based on the detected feature state change, the target map data identifying a location on the surface of interest within the patient's body.

16. The system of claim 15, wherein the target generator is to modify a graphical map for the patient to include a graphical element identifying the location on the surface of interest within the patient's body.

17. The system of claim 16, wherein the machine readable instructions comprise a state dynamic calculator to:
create a feature state matrix based on at least the feature states;
compute state dynamics based on the feature state matrix; and
predict a likelihood of procedure success based on the computed state dynamics.

18. A computer-implemented method comprising:
receiving, by a processor, electrophysiological data captured from a patient during a therapy treatment of a target site identified prior to a treatment, the target site corresponding to a potential ablation site on a surface of interest within a patient's body;
generating, by the processor, feature signals based on the electrophysiological data captured from the patient;
computing, by the processor, feature states based on the feature signals;
detecting, by the processor, a respective feature state change based on an evaluation of the feature states relative to state change detection criteria; and
outputting, by the processor, target map data identifying a region of interest on a surface of interest.

19. The computer-implemented method of claim 18, wherein the region of interest has signal feature values that are similar to signal feature values at a prior treated location which elicited a detected arrhythmia state change.

20. The computer-implemented method of claim 18, further comprising:
computing, by the processor, a number of feature values for each feature based on respective portions of electrophysiological signals of the electrophysiological data;
combining, by the processor, the feature values for each feature to provide the feature signals; and
computing, by the processor, the feature states based on a feature signal segment from one of the feature signals.
